(19) United States
(12) Patent Application Publication
(10) Pub. No.: US 2016/0009727 A1
(43) Pub. Date: Jan. 14, 2016

(54) SANGUINARINE ANALOG PP2C INHIBITORS FOR CANCER TREATMENT

(71) Applicants: John S. KOVACH, (US); Francis JOHNSON, (US); Ramakrishna SAMUDRALA, (US)

(72) Inventors: John S. KOVACH, East Setauket, NY (US); Francis JOHNSON, Setauket, NY (US); Ramakrishna SAMUDRALA, Port Jefferson Station, NY (US)

(73) Assignee: Lixte Biotechnology, Inc., East Setauket, NY (US)

(21) Appl. No.: 14/772,327

(22) PCT Filed: Feb. 27, 2014
(86) PCT No.: PCT/US14/18991

§ 371 Date: Sep. 2, 2015

Related U.S. Application Data
(60) Provisional application No. 61/794,565, filed on Mar. 15, 2013.

(51) Int. Cl. C07D 491/153

(52) U.S. Cl. CPC C07D 491/153

(57) ABSTRACT

Sanguinarine analogs as PP2C inhibitors are disclosed for the treatment of various cancers, as well as methods of synthesizing such analogs.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

Serine/threonine phosphatases have been shown to have important roles in growth regulation and in stress signaling in normal and cancer cells. One group, the PP2C proteins, constitute one family of 8 protein serine/threonine phosphatase families in eukaryotic cells (Shreeram and Bulavin, 2008; Lammers and Lavi, 2007). Of particular importance of these enzymes as potential anti-cancer targets is their ability to regulate aspects of the cell cycle, particularly steps essential to mitosis (Wurzenberger and Gerlach, 2011).

Despite the appeal of the PP2Cs as targets for cancer therapy, there is a paucity of small molecules with inhibitory activity against this class of molecules. Recently, Aburai et al (2010) identified a naturally occurring plant alkaloid, sanguinarine (13-methyl-[1,3]benzodioxolo[5,6-c]-1,3-dioxolo[4,5-i]phenanthridinium), as an inhibitor of PP2C.

Sugiura et al. (2008) recently reported that a member of the protein phosphatase family (PPM1H) is increased in human colon adenocarcinomas and that reduction of PPM1H expression using small interfering RNAs decreased the growth of the human breast cancer cell line, MCF-7. Sun et al. (2012) showed that sanguinarine inhibits activation of the transcription factor Stat3 the growth of two human prostate cancer cell lines, that it may have potential as a therapeutic agent for prostate cancer. The possibility that sanguinarine may be valuable as an anti-cancer agent is further supported by the finding of Ahmed et al. (2010) that sanguinarine has greater inhibitory activity (anti-proliferative and apoptotic) against human cancer cell lines compared to normal human cells (epidermal keratinocytes).

### SUMMARY OF THE INVENTION

The subject invention provides a compound having the structure:

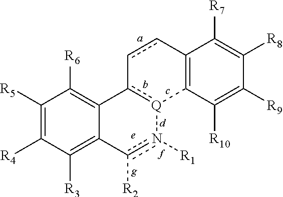

wherein:
bond “a” is a single bond or a double bond;
bond “b” is a single bond or a double bond;
bond “c” is a single bond which is present or absent;
bond “d” is a single bond which is present or absent;
bond “e” is a single bond, a double bond or a triple bond;
bond “f” is a single bond which is present or absent;
bond “g” is a single bond which is present or absent;

### Q is

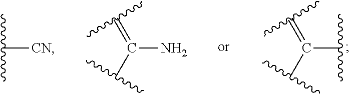

R1 is present or absent, wherein when R1 is present, then bond “f” is present, and when R1 is absent, then bond “f” is absent;
R2 is present or absent, wherein when R2 is present, then bond “g” is present, and when R2 is absent, then bond “g” is absent;
wherein when bond “d” is absent, then bond “a” is a single bond, bond “b” is a single bond, bond “c” is absent, bond “e” is a triple bond, R1 is absent, R2 is absent and Q is

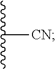 [SMILES: *C#N]

wherein when bond “d” is present, then bond “b” is a double bond, bond “e” is a single bond or a double bond and R2 is present;
wherein when bond “c” is absent and bond “d” is present, then bond “a” is a single bond, “e” is a double bond and Q is

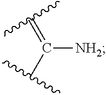 [SMILES: *C(=*)N]

wherein when bond “c” is present, then bond “e” is a single bond or a double bond and Q is

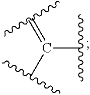 [SMILES: *C(*)=*]

wherein when bond “e” is a single bond, then bond “a” is a double bond and R1 is present;
wherein when R1 is present, then N is N⁺ and R1 is C1-C10 alkyl other than methyl, C2-C10 alkenyl, C3-C10 alkynyl, C3-C7 cycloalkyl, C2-C10 acyl, C2-C10 heteroalkyl, aryl or arylmethyl, any one of which is unsubstituted or substituted at one or more positions with halogen, C1-C5 alkyl, C1-C5 heteroalkyl, C2-C7 acyl, C3-C7 cycloalkyl or aryl;
wherein when R2 is present, then R2 is hydrogen, halogen or amino, wherein the amino is unsubstituted or substituted with one or more C1-C5 alkyl, C2-C7 acyl, aryl or arylmethyl;
wherein R3, R4, R5 and R6 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-monoalkylamino, C1-C5 dialkylamino, C2-C7 acylamino or arylmethylamino, wherein at least one of R3, R4, R5 and R6 are other than hydrogen, or R3 and R4 are linked so as to form —O—(CX₂)ₙ—O—, or R4 and R5 are linked so as to form —O—(CX₂)ₙ—O—, or R5 and R6 are linked so as to form —O—(CX₂)ₙ—O—, wherein X is hydrogen, methyl or fluorine and n is 1-2;
wherein R7, R8, R9 and R10 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R7, R8, R9 and R10 are other than hydrogen, or R7 and R8 are linked so as to form —O—(CX₂)ₙ—O—, R8 and R9 are linked so as to form —O—(CX₂)ₙ—O—, or R9 and R10 are linked so as to form —O—(CX₂)ₙ—O—, wherein X is hydrogen, methyl or fluorine and n is 1-2; and
wherein when bond “c” is present and R2 is hydrogen, then R1 is present, or
a pharmaceutically acceptable salt thereof.

The subject invention provides a method of inhibiting protein phosphatase 2C (PP2C) comprising contacting the PP2C with a compound having the structure:

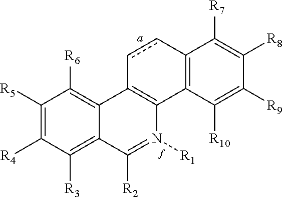

wherein bond “a” is a single bond or a double bond;
bond “f” is a single bond which is present or absent;
R1 is present or absent, wherein when R1 is present, then bond “f” is present, and when R1 is absent, then bond “f” is absent;
wherein when R1 is present, then N is N⁺ and R1 is C1-C10 alkyl other than methyl, C2-C10 alkenyl, C3-C10 alkynyl, C3-C7 cycloalkyl, C2-C10 acyl, C2-C10 heteroalkyl, aryl or arylmethyl, any one of which is unsubstituted or substituted at one or more positions with halogen, C1-C5 alkyl, C1-C5 heteroalkyl, C2-C7 acyl, C3-C7 cycloalkyl or aryl;
wherein when R2 is present, then R2 is hydrogen, halogen or amino, wherein the amino is unsubstituted or substituted with one or more C1-C5 alkyl, C2-C7 acyl, aryl or arylmethyl;
wherein R3, R4, R5 and R6 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-monoalkylamino, C1-C5 dialkylamino, C2-C7 acylamino or arylmethylamino, wherein at least one of R3, R4, R5 and R6 are other than hydrogen, or R3 and R4 are linked so as to form —O—(CX₂)ₙ—O—, or R4 and R5 are linked so as to form —O—(CX₂)ₙ—O—, or R5 and R6 are linked so as to form —O—(CX₂)ₙ—O—, wherein X is hydrogen, methyl or fluorine and n is 1-2;
wherein R7, R8, R9 and R10 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R7, R8, R9 and R10 are other than hydrogen, or R7 and R8 are linked so as to form —O—(CX₂)ₙ—O—, R8 and R9 are linked so as to form —O—(CX₂)ₙ—O—, or R9 and R10 are linked so as to form —O—(CX₂)ₙ—O—, wherein X is hydrogen, methyl or fluorine and n is 1-2; and
wherein when bond “c” is present and R2 is hydrogen, then R1 is present; or
a pharmaceutically acceptable salt thereof, so as to thereby inhibit the PP2C.

The subject invention provides a method of inhibiting growth of cells, comprising contacting cells with a compound having the structure:

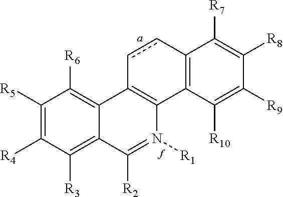

wherein bond “a” is a single bond or a double bond;
bond “f” is a single bond which is present or absent;
R1 is present or absent, wherein when R1 is present, then bond “f” is present, and when R1 is absent, then bond “f” is absent;
wherein when R1 is present, then N is N⁺ and R1 is C1-C10 alkyl other than methyl, C2-C10 alkenyl, C3-C10 alkynyl, C3-C7 cycloalkyl, C2-C10 acyl, C2-C10 heteroalkyl, aryl or arylmethyl, any one of which is unsubstituted or substituted at one or more positions with halogen, C1-C5 alkyl, C1-C5 heteroalkyl, C2-C7 acyl, C3-C7 cycloalkyl or aryl;
wherein when R2 is present, then R2 is hydrogen, halogen or amino, wherein the amino is unsubstituted or substituted with one or more C1-C5 alkyl, C2-C7 acyl, aryl or arylmethyl;
wherein R3, R4, R5 and R6 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R3, R4, R5 and R6 are other than hydrogen, or R3 and R4 are linked so as to form —O—(CX₂)ₙ—O—, or R4 and R5 are linked so as to form —O—(CX₂)ₙ—O—, or R5 and R6 are linked so as to form —O—(CX₂)ₙ—O—, wherein X is hydrogen, methyl or fluorine and n is 1-2;
wherein R7, R8, R9 and R10 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R7, R8, R9 and R10 are other than hydrogen, or R7 and R8 are linked so as to form —O—(CX₂)ₙ—O—, R8 and R9 are linked so as to form —O—(CX₂)ₙ—O—, or R9 and R10 are linked so as to form —O—(CX₂)ₙ—O—, wherein X is hydrogen, methyl or fluorine and n is 1-2; and
wherein when bond “c” is present and R2 is hydrogen, then R1 is present; or
a pharmaceutically acceptable salt thereof, so as to thereby inhibit growth of the cells.

The subject invention provides a method of treating cancer in a patient afflicted by the cancer, comprising administering to the patient a compound having the structure:

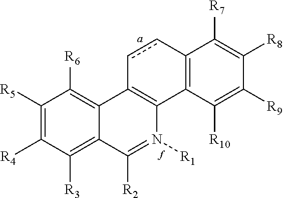

wherein bond “a” is a single bond or a double bond;
bond “f” is a single bond which is present or absent;
R1 is present or absent, wherein when R1 is present, then bond “f” is present, and when R1 is absent, then bond “f” is absent;
wherein when R1 is present, then N is N⁺ and R1 is C1-C10 alkyl other than methyl, C2-C10 alkenyl, C3-C10 alkynyl, C3-C7 cycloalkyl, C2-C10 acyl, C2-C10 heteroalkyl, aryl or arylmethyl, any one of which is unsubstituted or substituted at one or more positions with halogen, C1-C5 alkyl, C1-C5 heteroalkyl, C2-C7 acyl, C3-C7 cycloalkyl or aryl;
wherein when R2 is present, then R2 is hydrogen, halogen or amino, wherein the amino is unsubstituted or substituted with one or more C1-C5 alkyl, C2-C7 acyl, aryl or arylmethyl;
wherein R3, R4, R5 and R6 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-monoalkylamino, C1-C5 dialkylamino, C2-C7 acylamino or arylmethylamino, wherein at least one of R3, R4, R5 and R6 are other than hydrogen, or R3 and R4 are linked so as to form —O—(CX₂)ₙ—O—, or R4 and R5 are linked so as to form —O—(CX₂)ₙ—O—, or R5 and R6 are linked so as to form —O—(CX₂)ₙ—O—, wherein X is hydrogen, methyl or fluorine and n is 1-2;
wherein R7, R8, R9 and R10 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R7, R8, R9 and R10 are other than hydrogen, or R7 and R8 are linked so as to form —O—(CX₂)ₙ—O—, R8 and R9 are linked so as to form —O—(CX₂)ₙ—O—, or R9 and R10 are linked so as to form —O—(CX₂)ₙ—O—, wherein X is hydrogen, methyl or fluorine and n is 1-2; and
wherein when bond “c” is present and R2 is hydrogen, then R1 is present, or
a pharmaceutically acceptable salt thereof, so as to thereby treat the cancer.

The subject invention provides a process of preparing a compound comprising:

(a) reacting a first compound with a strong base and a second compound;
wherein the first compound has the structure:

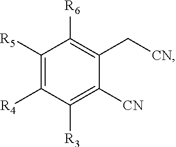

wherein R3, R4, R5 and R6 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R3, R4, R5 and R6 are other than hydrogen, or R3 and R4 are linked so as to form —O—(CX₂)ₙ—O—, or R4 and R5 are linked so as to form —O—(CX₂)ₙ—O—, or R5 and R6 are linked so as to form —O—(CX₂)ₙ—O—, wherein X is hydrogen, methyl or fluorine and n is 1-2;
wherein when R3, R4, R5 or R6 are hydroxyl, thiol or amino, R3, R4, R5 or R6 are optionally substituted with a suitable protecting group; and
wherein the second compound has the structure:

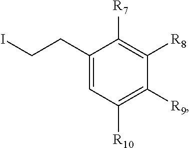

wherein R7, R8, R9 and R10 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R7, R8, R9 and R10 are other than hydrogen, or R7 and R8 are linked so as to form —O—(CX₂)ₙ—O—, R8 and R9 are linked so as to form —O—(CX₂)ₙ—O—, or R9 and R10 are linked so as to form —O—(CX₂)ₙ—O—, wherein X is hydrogen, methyl or fluorine and n is 1-2;
wherein when R7, R8, R9 or R10 are hydroxyl, thiol or amino, R7, R8, R9 or R10 are optionally substituted with a suitable protecting group;
to form a product having the structure:

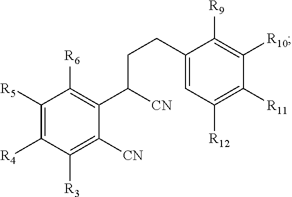

(b) reacting the product of step (a) with an acid to form a product having the structure:

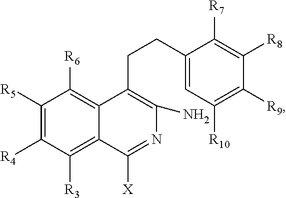

wherein X is C1, Br, I or trifluoromethanesulfonate;
(c) reacting the product of step (b) under hydrogenation conditions to form a product having the structure:

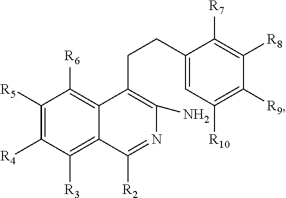

wherein R2 is hydrogen; and
(d) reacting the product of step (c) with sodium nitrite and acid, then heating to form a product having the structure:

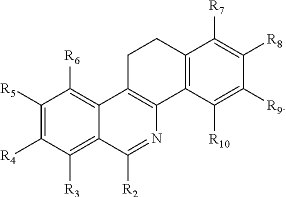

The subject invention provides a process of preparing a compound comprising:

(a) reacting a first compound with a strong base and a second compound;
wherein the first compound has the structure:

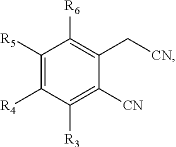

wherein R3, R4, R5 and R6 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R3, R4, R5 and R6 are other than hydrogen, or R3 and R4 are linked so as to form —O—(CX₂)ₙ—O—, or R4 and R5 are linked so as to form —O—(CX₂)ₙ—O—, or R5 and R6 are linked so as to form —O—(CX₂)ₙ—O—, wherein X is hydrogen, methyl or fluorine and n is 1-2;
wherein when R3, R4, R5 or R6 are hydroxyl, thiol or amino, R3, R4, R5 or R6 are optionally substituted with a suitable protecting group; and
wherein the second compound has the structure:

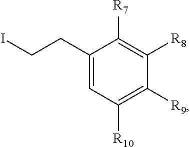

wherein R7, R8, R9 and R10 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R7, R8, R9 and R10 are other than hydrogen, or R7 and R8 are linked so as to form —O—(CX₂)ₙ—O—, R8 and R9 are linked so as to form —O—(CX₂)ₙ—O—, or R9 and R10 are linked so as to form —O—(CX₂)ₙ—O—, wherein X is hydrogen, methyl or fluorine and n is 1-2;
wherein when R7, R8, R9 or R10 are hydroxyl, thiol or amino, R7, R8, R9 or R10 are optionally substituted with a suitable protecting group;
to form a product having the structure:

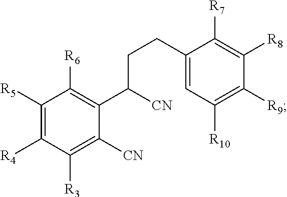

(b) reacting the product of step (a) by treating the product of step (a) with trifluoroacetic acid or fluoroboric acid to form a product having the structure:

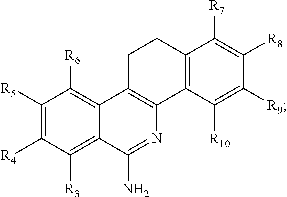

and
(c) reacting the product of step (b) under conditions so as to result in a product having the structure:

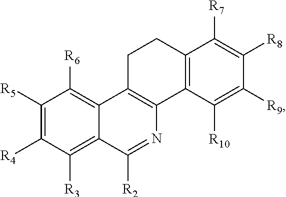

wherein R2 is hydrogen, halogen or amino, wherein the amino is unsubstituted or substituted with one or more C1-C5 alkyl, C2-C7 acyl, aryl or arylmethyl, or
a pharmaceutically acceptable salt thereof.

### BRIEF DESCRIPTION OF THE FIGURES

FIG. 1. Table summarizing the ability of sanguinarine, norsanguinarine (Compound 15), dihydrosanguinarine (Compound 14), the ethyl-substituted derivative (Compound 17), to inhibit growth in vitro of two human cancer cell lines, U87MG (glioblastoma) and A549 (lung carcinoma). The ethyl-substituted derivative (Compound 17) inhibited both cell lines with an ICH of about 3.3 and 1.6 μM respectively, whereas sanguinarine itself had IC₅₀ values of 2.0 and 0.8 μM, respectively.

FIG. 2. Chart showing the results of a first A549 WST assay comparing the effect of sanguinarine, norsanguinarine (Compound 15), dihydrosanguinarine (Compound 14), the ethyl-substituted derivative (Compound 17), and known, highly active, anti-cancer agent Topotecan. The chart shows that ethyl-substituted derivative (Compound 17) inhibits cell growth and reduces cell viability of A549 WST. The ethyl-substituted derivative (Compound 17) inhibits A459 with an ICH of about 1.6 uM, whereas sanguinarine itself had an ICH of 0.8 μM. Topotecan showed ICH of about 0.2 μM against the same cell line. Dihydrosanguinarine and norsanguinarine had no activity up to 10 μM.

FIG. 3. Chart showing the results of a second A549 WST assay comparing the effect of sanguinarine, norsanguinarine (Compound 15), dihydrosanguinarine (Compound 14), the ethyl-substituted derivative (Compound 17), and known, highly active, anti-cancer agent Topotecan. The chart shows that ethyl-substituted derivative

(Compound 17) inhibits cell growth and reduces cell viability of A549 WST. The ethyl-substituted derivative (Compound 17) inhibits A459 with an ICH of about 1.6 uM, whereas sanguinarine itself had an IC₅₀ of 0.8 μM. Topotecan showed IC₅₀ of about 0.2 μM against the same cell line. Dihydrosanguinarine and norsanguinarine had no activity up to 10 μM.

FIG. 4. Chart showing the results of a first U-87 MG WST Assay comparing the effect of sanguinarine, norsanguinarine (Compound 15), dihydrosanguinarine (Compound 14), the ethyl-substituted derivative (Compound 17), and known, highly active, anti-cancer agent Topotecan. The chart shows that ethyl-substituted derivative (Compound 17) inhibits cell growth and reduces cell viability of U-87 MG WST. The ethyl-substituted derivative (Compound 17) inhibits U-87 MG with an ICH of about 3.3 μM, whereas sanguinarine itself had an ICH values of 2.0 μM. Topotecan showed ICH of about 0.3 μM against the same cell line. Dihydrosanguinarine and norsanguinarine had no activity up to 10 μM.

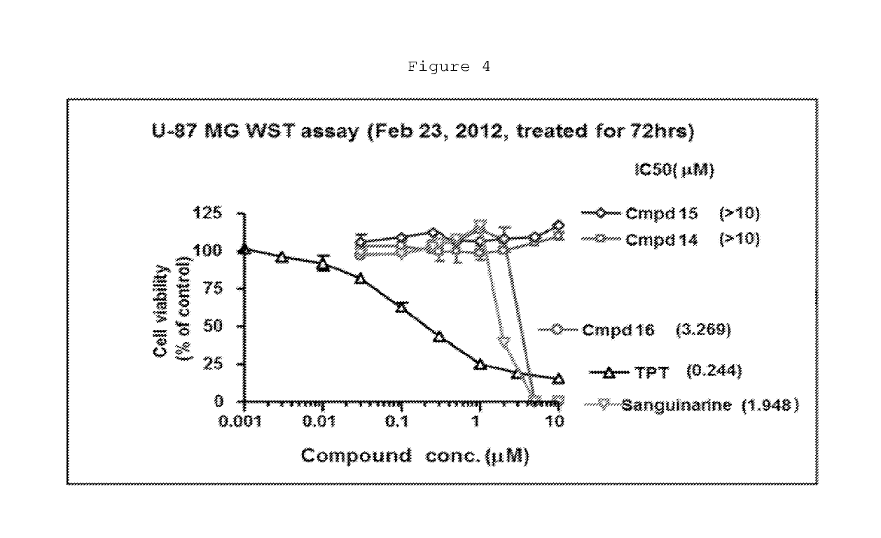

FIG. 5. Chart showing the results of a second U-87 MG WST Assay comparing the effect of sanguinarine, norsanguinarine (Compound 15), dihydrosanguinarine (Compound 14), the ethyl-substituted derivative (Compound 17), and known, highly active, anti-cancer agent Topotecan. The chart shows that ethyl-substituted derivative (Compound 17) inhibits cell growth and reduces cell viability of U-87 MG WST. The ethyl-substituted derivative (Compound 17) inhibits U-87 MG with an ICH of about 3.3 μM, whereas sanguinarine itself had an ICH values of 2.0 μM. Topotecan showed ICH of about 0.3 μM against the same cell line. Dihydrosanguinarine and norsanguinarine had no activity up to 10 μM.

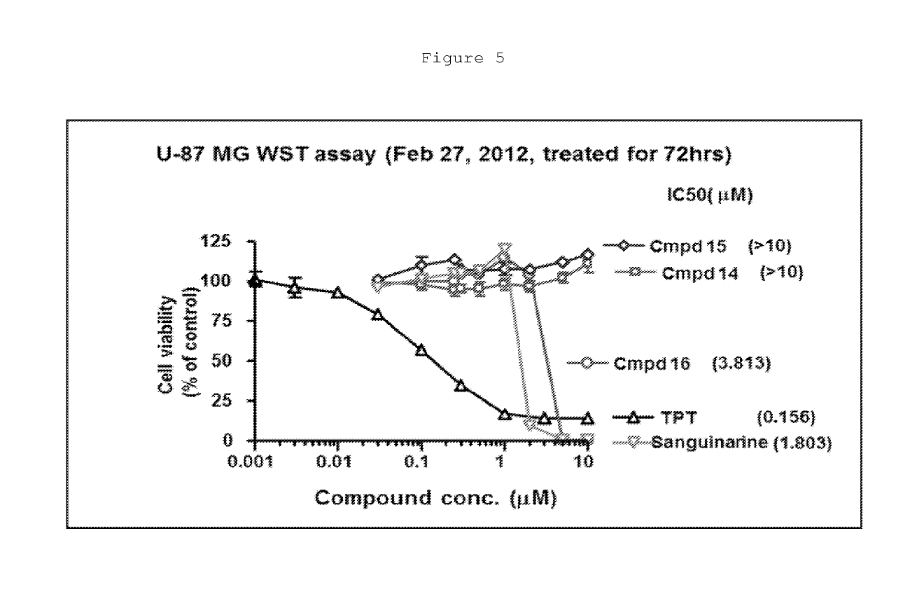

### DETAILED DESCRIPTION OF THE INVENTION

### What is Claimed is

The subject invention provides a compound having the structure:

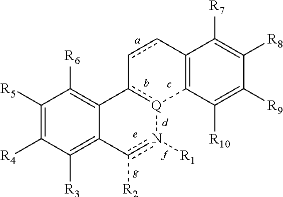

wherein:
bond “a” is a single bond or a double bond;
bond “b” is a single bond or a double bond;
bond “c” is a single bond which is present or absent;
bond “d” is a single bond which is present or absent;
bond “e” is a single bond, a double bond or a triple bond;
bond “f” is a single bond which is present or absent;
bond “g” is a single bond which is present or absent;

### Q is

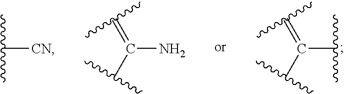

R1 is present or absent, wherein when R1 is present, then bond “f” is present, and when R1 is absent, then bond “f” is absent;
R2 is present or absent, wherein when R2 is present, then bond “g” is present, and when R2 is absent, then bond “g” is absent;
wherein when bond “d” is absent, then bond “a” is a single bond, bond “b” is a single bond, bond “c” is absent, bond “e” is a triple bond, R1 is absent, R2 is absent and Q is

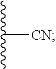 [SMILES: *C#N]

wherein when bond “d” is present, then bond “b” is a double bond, bond “e” is a single bond or a double bond and R2 is present;
wherein when bond “c” is absent and bond “d” is present, then bond “a” is a single bond, “e” is a double bond and Q is

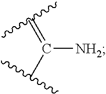 [SMILES: *C(=*)N]

wherein when bond “c” is present, then bond “e” is a single bond or a double bond and Q is

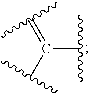 [SMILES: *C(*)=*]

wherein when bond “e” is a single bond, then bond “a” is a double bond and R1 is present;
wherein when R1 is present, then N is N⁺ and R1 is C1-C10 alkyl other than methyl, C2-C10 alkenyl, C3-C10 alkynyl, C3-C7 cycloalkyl, C2-C10 acyl, C2-C10 heteroalkyl, aryl or arylmethyl, any one of which is unsubstituted or substituted at one or more positions with halogen, C1-C5 alkyl, C1-C5 heteroalkyl, C2-C7 acyl, C3-C7 cycloalkyl or aryl;
wherein when R2 is present, then R2 is hydrogen, halogen or amino, wherein the amino is unsubstituted or substituted with one or more C1-C5 alkyl, C2-C7 acyl, aryl or arylmethyl;
wherein R3, R4, R5 and R6 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-monoalkylamino, C1-C5 dialkylamino, C2-C7 acylamino or arylmethylamino, wherein at least one of R3, R4, R5 and R6 are other than hydrogen, or R3 and R4 are linked so as to form —O—(CX₂)ₙ—O—, or R4 and R5 are linked so as to form —O—(CX₂)ₙ—O—, or R5 and R6 are linked so as to form —O—(CX₂)ₙ—O—, wherein X is hydrogen, methyl or fluorine and n is 1-2;
wherein R7, R8, R9 and R10 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R7, R8, R9 and R10 are other than hydrogen, or R7 and R8 are linked so as to form —O—(CX₂)ₙ—O—, R8 and R9 are linked so as to form —O—(CX₂)ₙ—O—, or R9 and R10 are linked so as to form —O—(CX₂)ₙ—O—, wherein X is hydrogen, methyl or fluorine and n is 1-2; and
wherein when bond “c” is present and R2 is hydrogen, then R1 is present, or
a pharmaceutically acceptable salt thereof.

In a further embodiment, the compound having the structure:

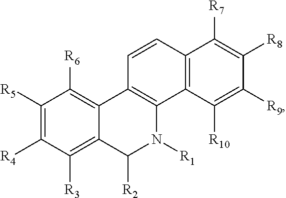

wherein R1 is present.

In a further embodiment, the compound having the structure:

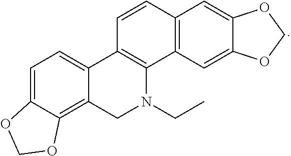 [SMILES: CCN1Cc2c(ccc3c2OCO3)-c2ccc3cc4c(cc3c21)OCO4]

In a further embodiment, the compound having the structure:

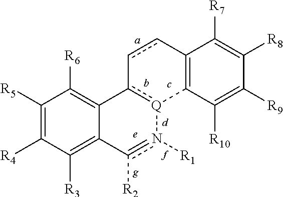

wherein:
bond “a” is a single bond or a double bond;
bond “b” is a single bond or a double bond;
bond “c” is a single bond which is present or absent;
bond “d” is a single bond which is present or absent;
bond “e” is a double bond or a triple bond;
bond “f” is a single bond which is present or absent;
bond “g” is a single bond which is present or absent;

### Q is

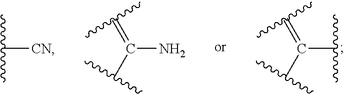

R1 is present or absent, wherein when R1 is present, then bond “f” is present, and when R1 is absent, then bond “f” is absent;
R2 is present or absent, wherein when R2 is present, then bond “g” is present, and when R2 is absent, then bond “g” is absent;
wherein when bond “d” is absent, then bond “a” is a single bond, bond “b” is a single bond, bond “c” is absent, bond “e” is a triple bond, R1 is absent, R2 is absent and Q is

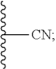 [SMILES: *C#N]

wherein when bond “d” is present, then bond “b” is a double bond, bond “e” is a double bond and R2 is present;
wherein when bond “c” is absent and bond “d” is present, then bond “a” is a single bond and Q is

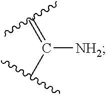 [SMILES: *C(=*)N]

wherein when bond “c” is present, then Q is

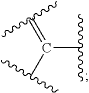 [SMILES: *C(*)=*]

wherein when R1 is present, then N is N⁺ and R1 is C1-C10 alkyl other than methyl, C2-C10 alkenyl, C3-C10 alkynyl, C3-C7 cycloalkyl, C2-C10 acyl, C2-C10 heteroalkyl, aryl or arylmethyl, any one of which is unsubstituted or substituted at one or more positions with halogen, C1-C5 alkyl, C1-C5 heteroalkyl, C2-C7 acyl, C3-C7 cycloalkyl or aryl;
wherein when R2 is present, then R2 is hydrogen, halogen or amino, wherein the amino is unsubstituted or substituted with one or more C1-C5 alkyl, C2-C7 acyl, aryl or arylmethyl;
wherein R3, R4, R5 and R6 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-monoalkylamino, C1-C5 dialkylamino, C2-C7 acylamino or arylmethylamino, wherein at least one of R3, R4, R5 and R6 are other than hydrogen, or R3 and R4 are linked so as to form —O—(CX₂)ₙ—O—, or R4 and R5 are linked so as to form —O—(CX₂)ₙ—O—, or R5 and R6 are linked so as to form —O—(CX₂)ₙ—O—, wherein X is hydrogen, methyl or fluorine and n is 1-2;
wherein R7, R8, R9 and R10 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R7, R8, R9 and R10 are other than hydrogen, or R7 and R8 are linked so as to form —O—(CX₂)ₙ—O—, R8 and R9 are linked so as to form —O—(CX₂)ₙ—O—, or R9 and R10 are linked so as to form —O—(CX₂)ₙ—O—, wherein X is hydrogen, methyl or fluorine and n is 1-2; and
wherein when bond “c” is present and R2 is hydrogen, then R1 is present, or
a pharmaceutically acceptable salt thereof.

In a further embodiment, the compound having the structure:

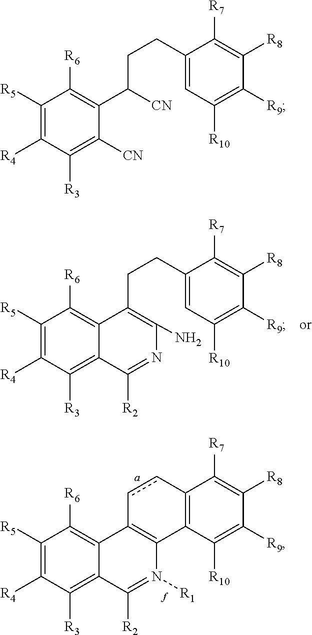

or
a pharmaceutically acceptable salt thereof.

In a further embodiment, the compound having the structure:

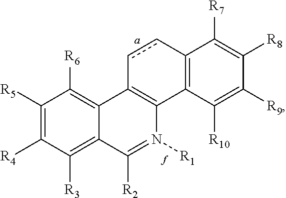

or
a pharmaceutically acceptable salt thereof.

In a further embodiment, the compound having the structure:

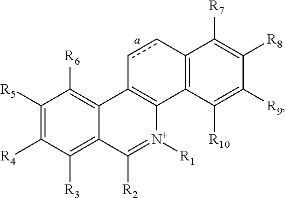

wherein R1 is present, or
a pharmaceutically acceptable salt thereof.

In a further embodiment, the compound wherein R8 and R9 are linked so as to form —O—CH₂—O—.

In a further embodiment, the compound wherein R3 and R4 are linked so as to form —O—CH₂—O—.

In a further embodiment, the compound wherein at least two of R3, R4, R5 and R6 are other than hydrogen; and at least two of R7, R8, R9 and R10 are other than hydrogen.

In a further embodiment, the compound wherein R5, R6, R7 and R10 are hydrogen.

In a further embodiment, the compound having the structure

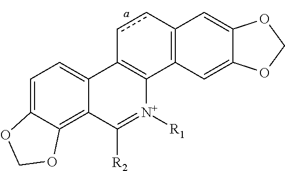

wherein R1 is present, or
a pharmaceutically acceptable salt thereof.

In a further embodiment, the compound wherein bond “a” is a single bond.

In a further embodiment, the compound having the structure

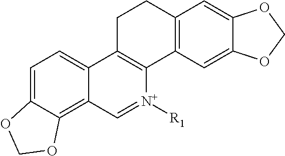

wherein R1 is present, or
a pharmaceutically acceptable salt thereof.

In a further embodiment, the compound wherein bond “a” is a double bond.

In a further embodiment, the compound having the structure

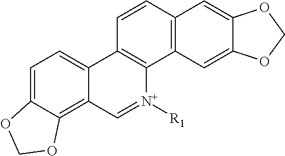

wherein R1 is present, or
a pharmaceutically acceptable salt thereof.

In a further embodiment, the compound wherein R1 is C1-C10 alkyl other than methyl, which is unsubstituted or substituted at one or more positions with halogen, C1-C5 alkyl, C1-C5 heteroalkyl, C2-C7 acyl, C3-C7 cycloalkyl or aryl.

In a further embodiment, the compound wherein R1 is not substituted.

In a further embodiment, the compound wherein R1 is a linear alkyl.

In a further embodiment, the compound wherein R1 is ethyl, propyl, butyl, pentyl, hexyl, heptyl, octyl, nonyl or decyl.

In a further embodiment, the compound wherein R1 is ethyl.

In a further embodiment, the compound wherein the compound is a pharmaceutically acceptable salt.

In a further embodiment, the compound wherein the pharmaceutically acceptable salt is a chloride, iodide, bromide, sulfate, bisulfate, nitrate, phosphate, sulfonate, formate, tartrate, maleate, malate, citrate, benzoate, acetate, valerate, oleate, palmitate, stearate, laurate, salicylate, ascorbate, tosylate, fumarate, succinate, napthylate, mesylate, glucoheptonate, lactobionate, laurylsulphonate or phenoate salt.

In a further embodiment, the compound wherein the pharmaceutically acceptable salt is a chloride, iodide or bromide.

In a further embodiment, the compound having the structure:

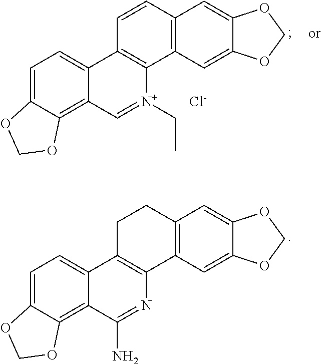 [SMILES: CC[n+]1cc2c3c(ccc2c2ccc4cc5c(cc4c21)OCO5)OCO3 ; [Cl-] ; Nc1nc2c(c3ccc4c(c13)OCO4)CCc1cc3c(cc1-2)OCO3]

In a further embodiment, the compound having the structure

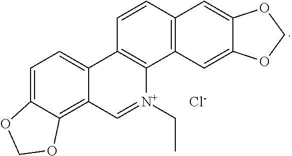 [SMILES: CC[n+]1cc2c3c(ccc2c2ccc4cc5c(cc4c21)OCO5)OCO3 ; [Cl-]]

In a further embodiment, the compound having the structure

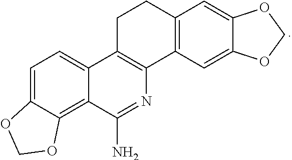 [SMILES: Nc1nc2c(c3ccc4c(c13)OCO4)CCc1cc3c(cc1-2)OCO3]

In a further embodiment, the compound having the structure:

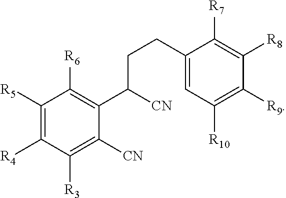

In a further embodiment, the compound having the structure:

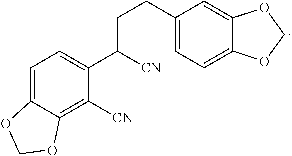 [SMILES: N#Cc1c(C(C#N)CCc2ccc3c(c2)OCO3)ccc2c1OCO2]

In a further embodiment, the compound having the structure:

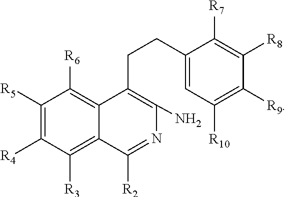

In a further embodiment, the compound having the structure:

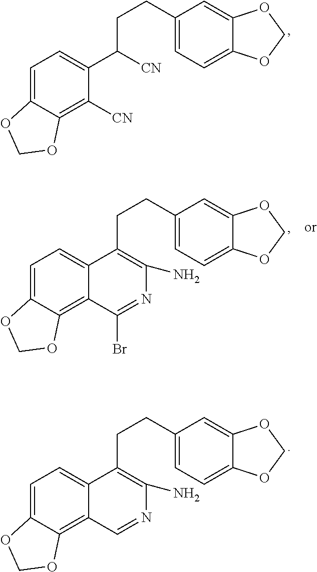 [SMILES: N#Cc1c(C(C#N)CCc2ccc3c(c2)OCO3)ccc2c1OCO2 ; Nc1nc(Br)c2c3c(ccc2c1CCc1ccc2c(c1)OCO2)OCO3 ; Nc1ncc2c3c(ccc2c1CCc1ccc2c(c1)OCO2)OCO3]

The subject invention provides a pharmaceutical composition comprising the compound of the subject invention.

The subject invention provides a method of inhibiting protein phosphatase 2C (PP2C) comprising contacting the PP2C with a compound having the structure:

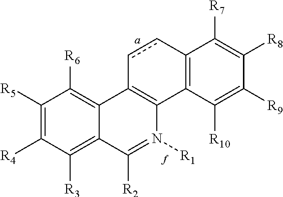

wherein bond “a” is a single bond or a double bond;
bond “f” is a single bond which is present or absent;
R1 is present or absent, wherein when R1 is present, then bond “f” is present, and when R1 is absent, then bond “f” is absent;
wherein when R1 is present, then N is N⁺ and R1 is C1-C10 alkyl other than methyl, C2-C10 alkenyl, C3-C10 alkynyl, C3-C7 cycloalkyl, C2-C10 acyl, C2-C10 heteroalkyl, aryl or arylmethyl, any one of which is unsubstituted or substituted at one or more positions with halogen, C1-C5 alkyl, C1-C5 heteroalkyl, C2-C7 acyl, C3-C7 cycloalkyl or aryl;
wherein when R2 is present, then R2 is hydrogen, halogen or amino, wherein the amino is unsubstituted or substituted with one or more C1-C5 alkyl, C2-C7 acyl, aryl or arylmethyl;
wherein R3, R4, R5 and R6 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R3, R4, R5 and R6 are other than hydrogen, or R3 and R4 are linked so as to form —O—(CX₂)ₙ—O—, or R4 and R5 are linked so as to form —O—(CX₂)ₙ—O—, or R5 and R6 are linked so as to form —O—(CX₂)ₙ—O—, wherein X is hydrogen, methyl or fluorine and n is 1-2;
wherein R7, R8, R9 and R10 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R7, R8, R9 and R10 are other than hydrogen, or R7 and R8 are linked so as to form —O—(CX₂)ₙ—O—, R8 and R9 are linked so as to form —O—(CX₂)ₙ—O—, or R9 and R10 are linked so as to form —O—(CX₂)ₙ—O—, wherein X is hydrogen, methyl or fluorine and n is 1-2; and
wherein when bond “c” is present and R2 is hydrogen, then R1 is present; or
a pharmaceutically acceptable salt thereof, so as to thereby inhibit the PP2C.

The subject invention provides a method of inhibiting growth of cells, comprising contacting cells with a compound having the structure:

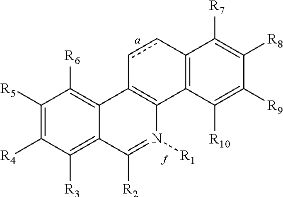

wherein bond “a” is a single bond or a double bond;
bond “f” is a single bond which is present or absent;
R1 is present or absent, wherein when R1 is present, then bond “f” is present, and when R1 is absent, then bond “f” is absent;
wherein when R1 is present, then N is N⁺ and R1 is C1-C10 alkyl other than methyl, C2-C10 alkenyl, C3-C10 alkynyl, C3-C7 cycloalkyl, C2-C10 acyl, C2-C10 heteroalkyl, aryl or arylmethyl, any one of which is unsubstituted or substituted at one or more positions with halogen, C1-C5 alkyl, C1-C5 heteroalkyl, C2-C7 acyl, C3-C7 cycloalkyl or aryl;
wherein when R2 is present, then R2 is hydrogen, halogen or amino, wherein the amino is unsubstituted or substituted with one or more C1-C5 alkyl, C2-C7 acyl, aryl or arylmethyl;
wherein R3, R4, R5 and R6 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-monoalkylamino, C1-C5 dialkylamino, C2-C7 acylamino or arylmethylamino, wherein at least one of R3, R4, R5 and R6 are other than hydrogen, or R3 and R4 are linked so as to form —O—(CX₂)ₙ—O—, or R4 and R5 are linked so as to form —O—(CX₂)ₙ—O—, or R5 and R6 are linked so as to form —O—(CX₂)ₙ—O—, wherein X is hydrogen, methyl or fluorine and n is 1-2;
wherein R7, R8, R9 and R10 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R7, R8, R9 and R10 are other than hydrogen, or R7 and R8 are linked so as to form —O—(CX₂)ₙ—O—, R8 and R9 are linked so as to form —O—(CX₂)ₙ—O—, or R9 and R10 are linked so as to form —O—(CX₂)ₙ—O—, wherein X is hydrogen, methyl or fluorine and n is 1-2; and
wherein when bond “c” is present and R2 is hydrogen, then R1 is present; or
a pharmaceutically acceptable salt thereof, so as to thereby inhibit growth of the cells.

In a further embodiment of the method, the cells are tumor cells.

In a further embodiment of the method, the cells are cancer cells.

In a further embodiment of the method, the cancer is lung cancer, breast cancer, prostate cancer, cervical cancer, pancreatic cancer, colon cancer, ovarian cancer; stomach cancer, esophagus cancer, mouth cancer, tongue cancer, gum cancer, skin cancer, muscle cancer, heart cancer, liver cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, gall bladder cancer, ocular cancer, joint cancer, glioblastoma, lymphoma, or leukemia.

In a further embodiment of the method, the compound inhibits the growth of cancer cells to a greater extent than normal cells.

The subject invention provides a method of treating cancer in a patient afflicted by the cancer, comprising administering to the patient a compound having the structure:

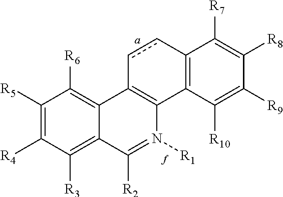

wherein bond “a” is a single bond or a double bond;
bond “f” is a single bond which is present or absent;
R1 is present or absent, wherein when R1 is present, then bond “f” is present, and when R1 is absent, then bond “f” is absent;
wherein when R1 is present, then N is N⁺ and R1 is C1-C10 alkyl other than methyl, C2-C10 alkenyl, C3-C10 alkynyl, C3-C7 cycloalkyl, C2-C10 acyl, C2-C10 heteroalkyl, aryl or arylmethyl, any one of which is unsubstituted or substituted at one or more positions with halogen, C1-C5 alkyl, C1-C5 heteroalkyl, C2-C7 acyl, C3-C7 cycloalkyl or aryl;
wherein when R2 is present, then R2 is hydrogen, halogen or amino, wherein the amino is unsubstituted or substituted with one or more C1-C5 alkyl, C2-C7 acyl, aryl or arylmethyl;
wherein R3, R4, R5 and R6 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-monoalkylamino, C1-C5 dialkylamino, C2-C7 acylamino or arylmethylamino, wherein at least one of R3, R4, R5 and R6 are other than hydrogen, or R3 and R4 are linked so as to form —O—(CX₂)ₙ—O—, or R4 and R5 are linked so as to form —O—(CX₂)ₙ—O—, or R5 and R6 are linked so as to form —O—(CX₂)ₙ—O—, wherein X is hydrogen, methyl or fluorine and n is 1-2;
wherein R7, R8, R9 and R10 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R7, R8, R9 and R10 are other than hydrogen, or R7 and R8 are linked so as to form —O—(CX₂)ₙ—O—, R8 and R9 are linked so as to form —O—(CX₂)ₙ—O—, or R9 and R10 are linked so as to form —O—(CX₂)ₙ—O—, wherein X is hydrogen, methyl or fluorine and n is 1-2; and
wherein when bond “c” is present and R2 is hydrogen, then R1 is present, or
a pharmaceutically acceptable salt thereof, so as to thereby treat the cancer.

In a further embodiment of the method, the compound bond “a” is a single bond.

In a further embodiment of the method, in the compound bond “a” is a double bond.

In a further embodiment of the method, in the compound R1 is present.

The subject invention provides a process of preparing a compound comprising:

(a) reacting a first compound with a strong base and a second compound;
wherein the first compound has the structure:

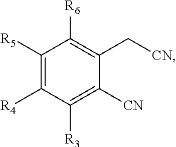

wherein R3, R4, R5 and R6 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R3, R4, R5 and R6 are other than hydrogen, or R3 and R4 are linked so as to form —O—(CX₂)ₙ—O—, or R4 and R5 are linked so as to form —O—(CX₂)ₙ—O—, or R5 and R6 are linked so as to form —O—(CX₂)ₙ—O—, wherein X is hydrogen, methyl or fluorine and n is 1-2;
wherein when R3, R4, R5 or R6 are hydroxyl, thiol or amino, R3, R4, R5 or R6 are optionally substituted with a suitable protecting group; and
wherein the second compound has the structure:

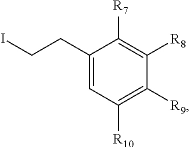

wherein R7, R8, R9 and R10 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R7, R8, R9 and R10 are other than hydrogen, or R7 and R8 are linked so as to form —O—(CX₂)ₙ—O—, R8 and R9 are linked so as to form —O—(CX₂)ₙ—O—, or R9 and R10 are linked so as to form —O—(CX₂)ₙ—O—, wherein X is hydrogen, methyl or fluorine and n is 1-2;
wherein when R7, R8, R9 or R10 are hydroxyl, thiol or amino, R7, R8, R9 or R10 are optionally substituted with a suitable protecting group;
to form a product having the structure:

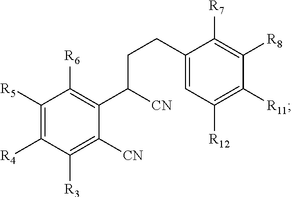

(b) reacting the product of step (a) with an acid to form a product having the structure:

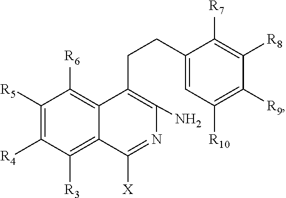

wherein X is C1, Br, I or trifluoromethanesulfonate;
(c) reacting the product of step (b) under hydrogenation conditions to form a product having the structure:

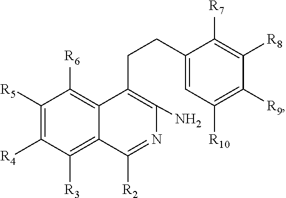

wherein R2 is hydrogen; and
(d) reacting the product of step (c) with sodium nitrite and acid, then heating to form a product having the structure:

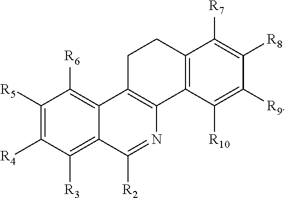

In a further embodiment, the process further comprising step (e) after step (d) said step (e) comprising:

(e) reacting the product of step (d) with platinum or palladium, and heating to form a product having the structure:

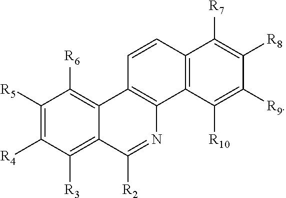

In a further embodiment, the process further comprising step (ee) after step (d) or (e), step (ee) comprising:

(ee) reacting the product of step (d) or (e) with a compound having the structure:

LG-R₁,

wherein LG is a leaving group; and
wherein R1 is C1-C10 alkyl other than methyl, C2-C10 alkenyl, C3-C10 alkynyl, C3-C7 cycloalkyl, C2-C10 acyl, C2-C10 heteroalkyl, aryl or arylmethyl, any one of which is unsubstituted or substituted at one or more positions with halogen, C1-C5 alkyl, C1-C5 heteroalkyl, C2-C7 acyl, C3-C7 cycloalkyl or aryl;
to form a product having the structure:

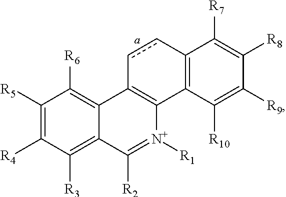

wherein when step (ee) is performed with the product of step (d), then bond “a” is a single bond, and when step (ee) is performed with the product of step (e), then bond “a” is a double bond, or
a pharmaceutically acceptable salt thereof.

In a further embodiment, the process further comprising step (f) after step (e), step (f) comprising:

(f) reacting the product of step (e) with sodium borohydride in the presence of a C1-C10 carboxylic acid, which is unsubstituted or substituted at one or more positions with C1-C5 alkyl, C1-C5 heteroalkyl, C3-C7 cycloalkyl or aryl;
to form a product having the structure:

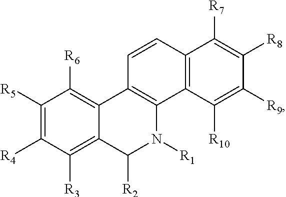

wherein R1 is C1-C10 alkyl, which is unsubstituted or substituted at one or more positions with C1-C5 alkyl, C1-C5 heteroalkyl, C3-C7 cycloalkyl or aryl.

In a further embodiment, the process further comprising step (g) after step (f), step (g) comprising:

(g) reacting the product of step (f) with chromium trioxide in dilute sulfuric acid and acetone to form a product having the structure:

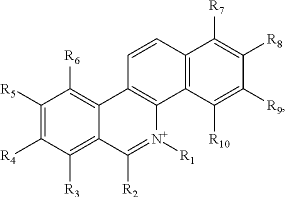

or a pharmaceutically acceptable salt thereof.

The subject invention provides a process of preparing a compound comprising:

(a) reacting a first compound with a strong base and a second compound;
wherein the first compound has the structure:

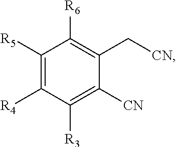

wherein R3, R4, R5 and R6 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-monoalkylamino, C1-C5 dialkylamino, C2-C7 acylamino or arylmethylamino, wherein at least one of R3, R4, R5 and R6 are other than hydrogen, or R3 and R4 are linked so as to form —O—(CX₂)ₙ—O—, or R4 and R5 are linked so as to form —O—(CX₂)ₙ—O—, or R5 and R6 are linked so as to form —O—(CX₂)ₙ—O—, wherein X is hydrogen, methyl or fluorine and n is 1-2;
wherein when R3, R4, R5 or R6 are hydroxyl, thiol or amino, R3, R4, R5 or R6 are optionally substituted with a suitable protecting group; and
wherein the second compound has the structure:

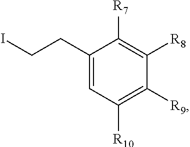

wherein R7, R8, R9 and R10 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R7, R8, R9 and R10 are other than hydrogen, or R7 and R8 are linked so as to form —O—(CX₂)ₙ—O—, R8 and R9 are linked so as to form —O—(CX₂)ₙ—O—, or R9 and R10 are linked so as to form —O—(CX₂)ₙ—O—, wherein X is hydrogen, methyl or fluorine and n is 1-2;
wherein when R7, R8, R9 or R10 are hydroxyl, thiol or amino, R7, R8, R9 or R10 are optionally substituted with a suitable protecting group;
to form a product having the structure:

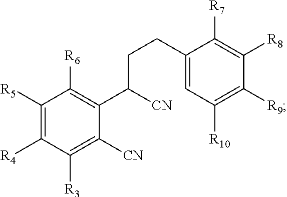

(b) reacting the product of step (a) by treating the product of step (a) with trifluoroacetic acid or fluoroboric acid to form a product having the structure:

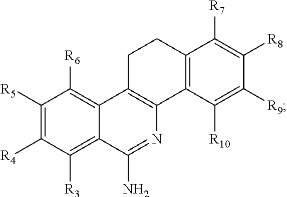

and
(c) reacting the product of step (b) under conditions so as to result in a product having the structure:

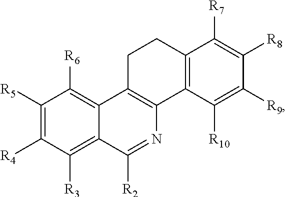

wherein R2 is hydrogen, halogen or amino, wherein the amino is unsubstituted or substituted with one or more C1-C5 alkyl, C2-C7 acyl, aryl or arylmethyl, or
a pharmaceutically acceptable salt thereof.

In a further embodiment, the process further comprising step (d) after step (c) said step (d) comprising:

(d) reacting the product of step (c) with platinum or palladium, and heating to form a product having the structure:

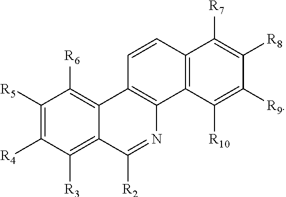

In a further embodiment, the process further comprising step (dd) after step (c) or (d), step (dd) comprising:

(dd) reacting the product of step (c) or (d) with a compound having the structure:

LG-R₁,

wherein LG is a leaving group; and
wherein R1 is C1-C10 alkyl other than methyl, C2-C10 alkenyl, C3-C10 alkynyl, C3-C7 cycloalkyl, C2-C10 acyl, C2-C10 heteroalkyl, aryl or arylmethyl, any one of which is unsubstituted or substituted at one or more positions with halogen, C1-C5 alkyl, C1-C5 heteroalkyl, C2-C7 acyl, C3-C7 cycloalkyl or aryl;
to form a product having the structure:

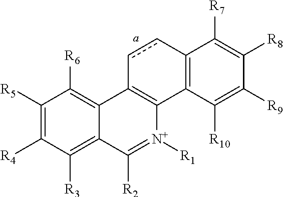

wherein when step (dd) is performed with the product of step (c), then bond “a” is a single bond, and when step (dd) is performed with the product of step (d), then bond “a” is a double bond, or
a pharmaceutically acceptable salt thereof.

In a further embodiment, the process further comprising step (e) after step (d), step (e) comprising:

(e) reacting the product of step (d) with sodium borohydride in the presence of a carboxylic acid having between 1 and 10 carbons, which is unsubstituted or substituted at one or more positions with C1-C5 alkyl, C1-C5 heteroalkyl, C3-C7 cycloalkyl or aryl;
to form a product having the structure:

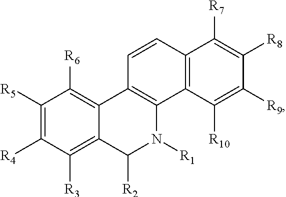

wherein R1 is C1-C10 alkyl, which is unsubstituted or substituted at one or more positions with C1-C5 alkyl, C1-C5 heteroalkyl, C3-C7 cycloalkyl or aryl.

In a further embodiment, the process further comprising step (f) after step (e), step (f) comprising:

(f) reacting the product of step (e) with chromium trioxide in dilute sulfuric acid and acetone to form a product having the structure:

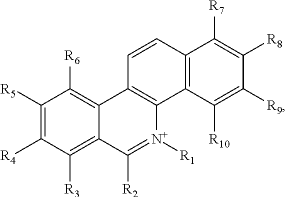

or a pharmaceutically acceptable salt thereof.

In a further embodiment, the process wherein LG is chloride.

In a further embodiment, the process wherein R1 is ethyl.

In a further embodiment, the process wherein R3 and R4 are linked so as to form —O—CH₂—O—.

In a further embodiment, the process wherein R5, R6, R7 and R10 are hydrogen.

In a further embodiment, the process wherein R8 and R9 are linked so as to form —O—CH₂—O—.

In a further embodiment, the process wherein the carboxylic acid is acetic acid.

Except where otherwise specified, when the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, N Y, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy(phenylmethoxy) and p-trifluoromethylbenzyloxy(4-trifluoromethylphenylmethoxy); heteroaryloxy groups; sulfonyl groups, such as trifluoromethanesulfonyl, methane sulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

In the compounds of the present invention, the substituents may be substituted or unsubstituted, unless specifically defined otherwise.

In the compounds of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds of the present invention, one of ordinary skill in the art will recognize that the various substituents, i.e. R₁, R₂, etc. are to be chosen in conformity with well-known principles of chemical structure connectivity.

As used herein, “C1-C10 alkyl” includes any linear or branched alkyl group having one to ten carbons. The alkyl group may be substituted or unsubstituted, including substituents which add additional carbons. For example, an unsubstituted “C1” is a methyl. “C1-C10 alkyl” includes alkyl groups having one, two, three, four, five, six, seven, eight, nine or ten carbon. The term “C1-C10” alkyl includes C1, C2, C3, C4, C5, C6, C7, C8, C9, and C10 alkyl. Alkyl groups corresponding to other ranges of sizes, such as C3-C7 alkyl or C1-C5 alkyl, are understood in the same manner as “C1-C10 alkyl” except pertaining to alkyl groups having a different number of carbons. For example, the term “C3-C10 alkyl” is any linear or branched alkyl group having three to ten carbons. The term “C2-C10” alkyl also includes all subsets of alkyl groups contained within including, but not limited to, C2-C4 alkyl, C5-C7 alkyl, C8-C10 alkyl, C2-C6 alkyl, C4-C8 alkyl, C6-C10 alkyl, C3-C10 alkyl, etc. Examples of “C1-C10 alkyl” include, but are not limited to, methyl, ethyl, n-propyl, isopropyl, n-butyl, t-butyl, pentyl, neopentyl, hexyl, and octyl. Unless otherwise specified, alkyl groups can be unsubstituted or substituted with one or more substituents, including but not limited to halogen, alkoxy, alkylthio, trifluoromethyl, difluoromethyl, methoxy, and hydroxyl.

As used herein, “C2-C10 alkenyl” is a C2-C10 alkyl group which contains one or more double bonds at any position. The double bonds may be internal double bonds or a terminal double bonds. The double bonds may be endo or exo double bonds. The double bonds may be cis or trans double bonds. The double bonds may be E or Z double bonds. The double bonds may be the result of tautomerism with another functional group. Examples of C2-C10 alkenyl include, but are not limited to, prop-2-en-1-yl, but-3-en-1-yl, 2,2-dimethyl-oct-4-en-1-yl, pent-4-en-1-yl.

As used herein, “C3-C10 alkynyl” is a C3-C10 alkyl group which contains one or more triple bonds at any position. The triple bonds may be internal triple bonds or a terminal triple bonds. Examples of C3-C10 alkynyl include, but are not limited to, prop-2-yn-1-yl, but-3-yn-1-yl, 2,2-dimethyl-oct-5-yn-1-yl, pent-4-yn-1-yl.

As used herein, “a carboxylic acid having between 1 and 10 carbons” is a C1-C10 linear or branched hydrocarbon comprising a carboxylic acid functionality at a terminal end so as to result in a hydrocarbon group having between one and ten carbon atoms. The carbonxylic acid may contain saturated or unsaturated rings, including phenyl rings. The carboxylic acid having between 1 and 10 carbons may be substituted or unsubstituted, including substituents which add additional carbons.
As used herein, “C3-C7 cycloalkyl” includes any alkyl group having three to seven carbons and containing a saturated hydrocarbon ring. The alkyl group may contain a cyclic alkyl portion and any number of branching or linear alkyl portions so as to result in a hydrocarbon group containing three to seven carbons. The cyclic alkyl may have any number of double bonds which may be endo or exo to the having The alkyl group may be substituted or unsubstituted, including substituents which add additional carbons. Examples of “C3-C7 cycloalkyl” include, but are not limited to, cyclopropyl, cyclopropylmethyl, cyclohexyl and 2-methylcycloprop-1-yl.

As used herein, “C2-C10 acyl” is a C2-C10 alkyl group having a ketone at the first position. “C2-C10 acyl” includes, but is not limited to, acetyl and benzoyl.

As used herein, “C2-C10 heteroalkyl” is a C2-C10 alkyl group wherein one or more of the carbons in the C2-C10 alkyl group are replaced by an oxygen, nitrogen or sulfur. “C2-C10 heteroalkyl” includes, but is not limited to, methoxyethyl, ethoxyethyl, ethoxyethoxyethyl 2-aminoethyl and 4-mercaptohex1-yl.

As used herein: “arylmethyl” is methyl group substituted with an aryl; “C1-C5 alkoxy” is an oxygen substituted with a C1-C5 alkyl; “C2-C7 acyloxy” is an oxygen substituted with a C2-C7 acyl group; “aryloxy” is an oxygen substituted with an aryl group; “arylmethyloxy” is an oxygen substituted with an arylmethyl group; “C1-C5 alkylthiol” is a sulfur substituted with a C1-C5 alkyl; “C2-C7 acylthiol” is a sulfur substituted with a C2-C7 acyl group; “arylthiol” is a sulfur substituted with an aryl group; “arylmethylthiol” is a sulfur substituted with an arylmethyl group; “C1-C5 monoalkylamino” is an amino group substituted with one C1-C5 alkyl group; “C1-C5 dialkylamino” is an amino group substituted with two C1-C5 alkyl groups; “C2-C7 acylamino” is an amino group substituted with one C2-C7 acyl group; “arylmethylamino” is an amino group substituted with an arylmethyl group.

As used herein, a “strong base” is a base that ionizes completely in aqueous solution.

As used herein, a “strong acid” is an acid that ionizes completely in aqueous solution.

As used herein, a “leaving group” is substituent which is displaced upon substitution at the substituent carbon. Examples of substituents which are leaving groups include, but are not limited to, diazonium salt, oxonium ion, nonaflate, triflate, fluorosulfonate, tosylate, mesylate, iodide, bromide, —OH₂⁺, chloride, tetraalkylammonium salts, fluorides, carboxylates, phenoxides and alkoxide.

As used herein, “monocycle” includes any stable polycyclic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polycyclic carbon ring of up to 10 atoms that is fused to a polycyclic carbon ring of up to atoms with each ring being independently unsubstituted or substituted. Examples of such non-aromatic bicycle elements include but are not limited to: decahydronaphthalene. Examples of such aromatic bicycle elements include but are not limited to: naphthalene.

As used herein, “aryl” is intended to “carbocyclic aryl” and “heterocyclic aryl”.

As used herein, “carbocyclic aryl” is intended to mean any stable monocyclic, bicyclic or polycyclic carbon ring of up to 10 atoms in each ring, wherein at least one ring is aromatic, and may be unsubstituted or substituted. Examples of such carbocyclic aryl elements include but are not limited to: phenyl, p-toluenyl(4-methylphenyl), naphthyl, tetrahydro-naphthyl, indanyl, phenanthryl, anthryl or acenaphthyl. In cases where the carbocyclic aryl substituent is bicyclic and one ring is non-aromatic, it is understood that attachment is via the aromatic ring.

As used herein, “heterocyclic aryl”, “heteroaryl” or “heterocycle”, is intended to mean a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include but are not limited to phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, triazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

As used herein, a “suitable protecting group” is a substituent group used to temporarily or selectively block the reactivity of a given functional group. A suitable protecting group includes, but is not limited to, a hydroxyl protecting group, a thiol protecting group or an amino protecting group.

“Hydroxyl protecting group” refers to a group blocking the OH function for subsequent reactions and can be removed under controlled conditions. Hydroxyl protecting groups are well known in the art, representative protecting groups are:—silyl ethers of formula —Si(R′)3, such as trimethylsilyl ether, triethylsilyl ether, tert-butyldimethylsilyl ether, tert-butyldiphenylsilyl ether, tri-isopropylsilyl ether, diethylisopropylsilyl ether, thexyldimethylsilyl ether, triphenylsilyl ether, di-tert-butylmethylsilyl ether;—alkyl and arylalkyl ethers such as methyl ether, tert-butyl ether, benzyl ether, p-methoxybenzyl ether, 3,4-dimethoxybenzyl ether, trityl ether; allyl ether;—alkoxymethyl and aryloxy ethers of formula —CH₂—O—R′, such as methoxymethyl ether, 2-methoxyethoxymethyl ether, benzyloxymethyl ether, p-methoxybenzyloxymethyl ether, 2-(trimethylsilyl)ethoxymethyl ether; tetrahydropyranyl and related ethers; methylthiomethyl ether;—esters of formula —C(═O)R′ such as acetate ester, benzoate ester; pivalate ester, methoxyacetate ester, chloroacetate ester, levulinate ester;—carbonates of formula —C(═O)—O—R′ such as benzyl carbonate, p-nitrobenzyl carbonate, tert-butyl carbonate, 2,2,2-trichloroethyl carbonate, 2-(trimethylsilyl)ethyl carbonate, allyl carbonate; and—sulphates such as SO₃.py. In all the above formula R′ represents a group selected from the group consisting of substituted or unsubstituted alkyl, substituted or unsubstituted alkenyl, substituted or unsubstituted alkynyl, substituted or unsubstituted aryl and substituted or unsubstituted arylalkyl. Additional examples of hydroxyl protecting groups can be found in reference books such as Greene and Wuts “Protective Groups in Organic Synthesis”, John Wiley & Sons, Inc., New Jersey, 2007.

“Thiol protecting group” refers to a group blocking the SH function for subsequent reactions and can be removed under controlled conditions. Thiol protecting groups are well known in the art. Representative protecting groups correspond to thioether and thioester analogues of the hydroxyl protecting groups listed above. Additional examples of thiol protecting groups can be found in reference books such as Greene and Wuts “Protective Groups in Organic Synthesis”, John Wiley & Sons, Inc., New Jersey, 2007.

“Amino protecting group” refers to a group blocking the NH₂ function for subsequent reactions and can be removed under controlled conditions. Amino protecting groups are well known in the art, representative protecting groups are carbamates, e.g. carbamates of formula —C(═O)OR′; amides, e.g. amides of formula —C(═O)R′, such as substituted or unsubstituted acetates; or silyl moieties of formula —Si(R′)3; wherein R′ is as defined above. Different alkyl moeties can also serve as amino protecting groups. Said alkyl groups can optionally be substituted with one or more substituents such as halogen, hydroxyl, alkoxyl, alkyloxymethyl ethers, carboxy, cyano, carbonyl, acyl, alkoxycarbonyl, amino, nitro, mercapto and alkylthio. Additional examples of amino protecting groups can be found in reference books such as Greene and Wuts “Protective Groups in Organic Synthesis”, John Wiley & Sons, Inc., New Jersey, 2007.

The compounds of the present invention may be prepared by techniques well know in organic synthesis and familiar to a practitioner ordinarily skilled in the art. However, these may not be the only means by which to synthesize or obtain the desired compounds.

The compounds of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5′ Edition (1996), March's Advanced Organic Chemistry: Reactions, Mechanisms, and Structure, Michael B. Smith, Jerry March, (Wiley-Interscience) 5′ Edition (2007), and references therein, which are incorporated by reference herein. However, these may not be the only means by which to synthesize or obtain the desired compounds.

The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

Another aspect of the invention is a pharmaceutical composition comprising the compound of the present invention.

Another aspect of the invention comprises a compound used in the method of the present invention as a pharmaceutical composition.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, “treating” means preventing, slowing, halting, or reversing the progression of a disease or infection. Treating may also mean improving one or more symptoms of a disease or infection.

The compounds of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds of the present invention may comprise a single compound or mixtures thereof with additional antibacterial agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of infection, all using dosage forms well known to those of ordinary skill in the pharmaceutical arts.

The compounds of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds of the present invention may also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxy-ethylaspartamidephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Method for the Total Synthesis of the N-Alkyl Sanguinarines as Illustrated by the Synthesis of N-Ethyl Sanguinarine

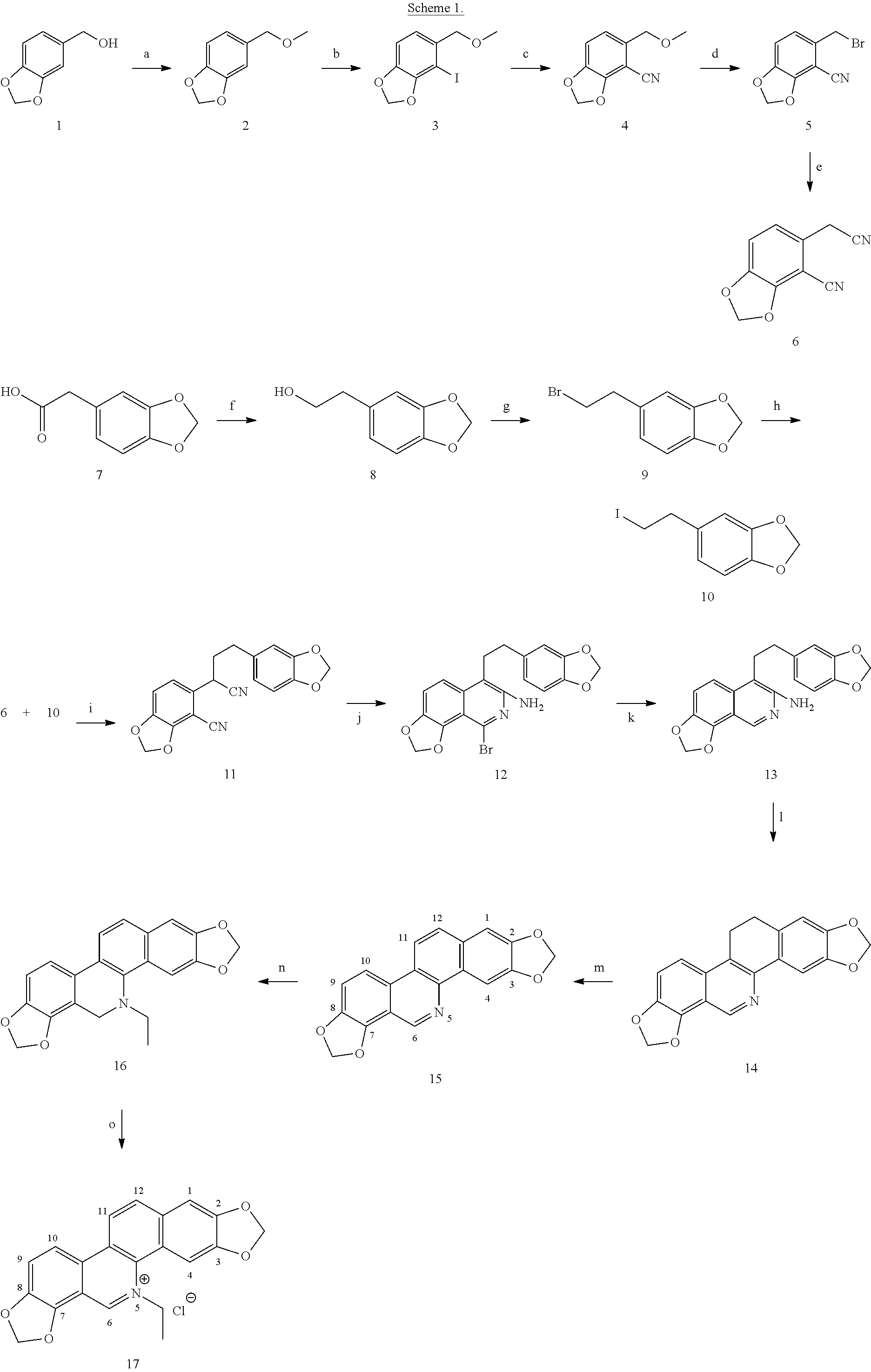

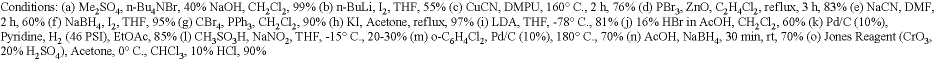

In order to prepare various analogues of sanguinarine, the tetracyclic norsanguinarine (15) was synthesized first, and the N-ethyl sanguinarine (17) was achieved as shown in Scheme 1. The two critical intermediates 6 and 10 that were needed were synthesized individually then coupled together to obtain the basic skeleton of sanguinarine (11).

Compound 6 was synthesized starting from the economical and commercially-available piperonyl alcohol (1). This alcohol 1 was methylated using dimethyl sulfate to give 2 in quantitative yield. Compound 2 was then treated with iodine in presence of n-BuLi to give compound 3 via directed ortho lithiation (DOM) reaction in a yield of 55%. The iodide 3 was then converted to 4 by heating with Cu(I)CN in DMPU. The resulting nitrile 4 was further treated with phosphorus tribromide and ZnO (catalytic amounts) to obtain the bromo compound 5 in 83% yield. This reaction was initially attempted by treating the methoxy compound 4 with PBr₃ in various halogenated solvents such as chloroform, dichloromethane and dichloroethane without ZnO and did not produce the required material. Hence a catalytic amount of ZnO was utilized to produce the required material in higher yields and in a short period of time. This reaction follows a Friedel-Crafts-type mechanism. Thereafter the bromide 5 was treated with NaCN in DMF to give the dicyano compound 6 in 60% yield. A significant amount of self-coupled side product was also produced in this reaction. Further work is needed to improve this step.

Compound 10 was prepared in three steps starting from the commercially available 3,4-methylenedioxyphenylacetic acid 7 following the procedures of Hurvois (Saurabh Shahane et al.). Compound 7 was reduced to alcohol 8 using NaBH₄ and I₂, which was further converted to bromide 9 then to iodide 10 under standard conditions.

In an attempt to prepare alkylated dicyano compound 11, compound 6 was initially treated with bromide 9 in the presence of bases such as NaH and LDA under different various reaction conditions. However none of these gave the required product. Bromide 9 remained intact in all trial reactions but the dicyano compound 6 decomposed. Subsequently, 9 was converted into a more reactive iodide 10 which on reaction with 6 in the presence of LDA gave the required mono alkylated dicyano compound 11 in 81% yields. The compound 11 was then treated with HBr in AcOH to give cyclized amino compound 12 following the conditions of Johnson et al (Johnson et al). In this reaction the aliphatic nitrile is protonated and this nitrilium ion attacks the aromatic nitrile causing ring-closure and the intermediate iminium ion traps a bromide ion. The bromo derivative 12 was then de-brominated using Pd/C (10%)/H₂ (46 PSI) in EtOAc in the presence of pyridine to give 13. Several attempts to convert this compound to 14 via an acid catalyzed cyclization were unsuccessful. Finally compound 13 was diazotized with sodium nitrite and methanesulfonic acid in THF to give 14. After diazotization the reaction mixture heated to generate a carbocation by losing N₂. This carbocation then cyclizes to the desired tetracyclic compound 14. This diazotization reaction was performed in various other acids also such as trifluoroacetic acid, acetic acid, sulfuric acid, and trifluoromethanesulfonic acid. However methanesulfonic acid was found to be giving better yields and less complex reaction mixtures. Dihydro compound 14 was then aromatized by heating in dichlorobenzene with Pd/C (10%) at 180 C to give norsanguinarine 15, following the conditions of Hibino (Hibino et. al).

In order to convert 15 into the desired N-ethyl sanguinarine derivative 17, we attempted a reaction on 15 with the reagents such as diethyl sulfate and ethyl iodide under various reaction conditions. However, none of these gave the desired product. It has been reported by Ishii et al′ that similar compounds such as O-ethyldecarine (18) can be converted into N,O-diethyldecarinium (20) by reductive N-alkylation followed by Jones oxidation as shown in the scheme below.
In order to convert 15 into the desired N-ethyl sanguinarine derivative 17, we attempted a reaction on 15 with the reagents diethyl sulfate and ethyl iodide under various reaction conditions.

However, none of these gave the desired product. It has been reported by Ishii et al⁴ that similar compounds such as O-ethyldecarine (18) can be converted into N,O-diethyldecarinium (20) by reductive N-alkylation followed by Jones oxidation as shown in the scheme below.

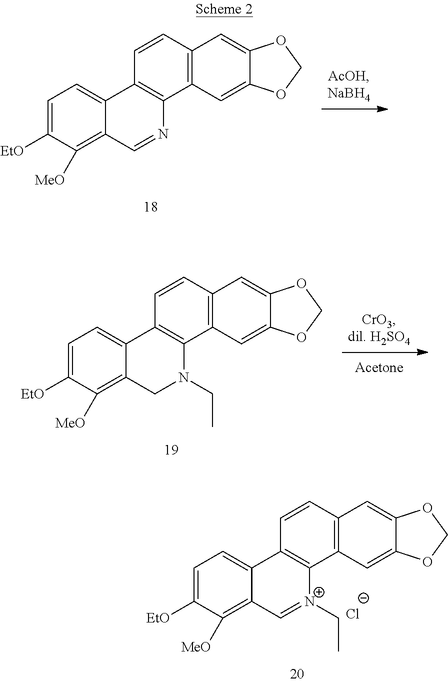 [SMILES: CCOc1ccc2c(cnc3c4cc5c(cc4ccc23)OCO5)c1OC ; CCOc1ccc2c(c1OC)CN(CC)c1c-2ccc2cc3c(cc12)OCO3 ; CCOc1ccc2c(c[n+](CC)c3c4cc5c(cc4ccc23)OCO5)c1OC ; [Cl-]]

Compound 15 was reacted with sodium borohydride in the presence of acetic acid to produce compound 16 in 70% yield. Compound 16 was reacted with Jones oxidation conditions (Chromium trioxide in dilute sulfuric acid and acetone) to produce compound 17 in 90% yield.

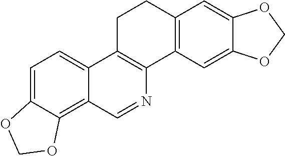 [SMILES: c1c2c(cc3c1OCO3)-c1ncc3c4c(ccc3c1CC2)OCO4]

### Compound 14 (dihydronorsanguinarine)-11,12-Dihydro-2,3,7,8-bismethylenedioxybenzo[c]phenanthridine

mp 246-247° C. ¹H NMR (300 MHz, CDCl₃) δ: 2.91-2.96 (2H, m), 3.20-3.25 (2H, m), 5.98 (2H, s), 6.24 (2H, s), 6.75 (1H, s), 7.38 (1H, d, J=8.7), 7.58 (1H, d, J=9.0), 7.89 (1H, s), 9.23 (1H, s).

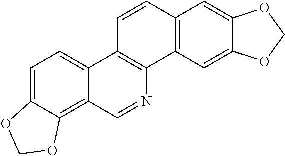 [SMILES: c1cc2c(cnc3c4cc5c(cc4ccc23)OCO5)c2c1OCO2]

### Compound 15 (norsanguinarine) 2,3,7,8-bismethylene-dioxybenzo[c]-phenanthridine (norsanguinarine)

mp 270-272° C. ¹H NMR (300 MHz, DMSO-d₆) δ: 6.22 (2H, s), 6.39 (2H, s), 7.53 (1H, s), 7.69 (1H, d, J=9.0), 7.99 (1H, d, J=9.0), 8.42 (1H, d, J=9.6), 8.51 (1H, s), 8.56 (1H, d, J=9.3), 9.41 (1H, s).

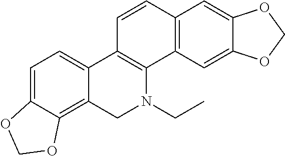 [SMILES: CCN1Cc2c(ccc3c2OCO3)-c2ccc3cc4c(cc3c21)OCO4]

### Compound 16—N-ethyl dihydrosanguinarine

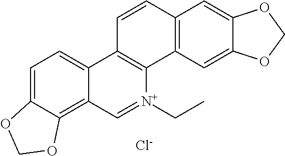 [SMILES: CC[n+]1cc2c3c(ccc2c2ccc4cc5c(cc4c21)OCO5)OCO3 ; [Cl-]]

### Compound 17—N-Ethyl Derivative of Sanguinarine

mp 258-259° C. ¹H NMR (300 MHz, CD₃OD) δ: 1.80 (3H, t, J=7.2), 5.32 (2H, q, J=6.9, 7.2), 6.27 (2H, s), 6.53 (2H, s), 7.58 (1H, s), 7.96 (2H, d, J=8.4), 8.23 (1H, d, J=9.0), 8.54 (1H, d, J=9.0), 8.64 (1H, d, J=9.0), 10.01 (1H, s).

### Example 2

### Alternative Synthesis of Dihydronorsanguinarine and Norsanguinarine

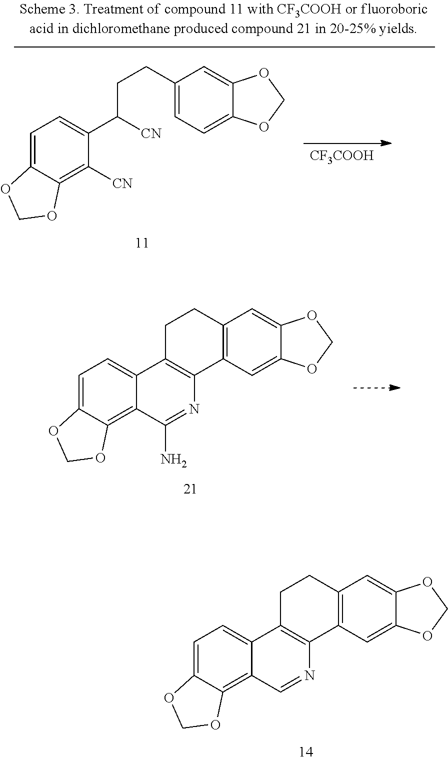 [SMILES: N#Cc1c(C(C#N)CCc2ccc3c(c2)OCO3)ccc2c1OCO2 ; "CF3COOH" ; Nc1nc2c(c3ccc4c(c13)OCO4)CCc1cc3c(cc1-2)OCO3 ; c1c2c(cc3c1OCO3)-c1ncc3c4c(ccc3c1CC2)OCO4]

### Example 3

### N-Alkyl Derivatives of Dihydrosanguinarine

N-alkyl derivatives of dihydrosanguinarine are prepared from reacting norsanguinarine (compound 15) with sodium borohydride in the presence of a carboxylic acid reagent (Table 1).

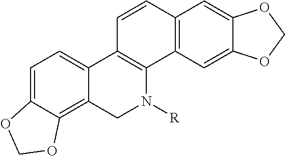

TABLE 1
Products resulting from reductive alkylation of norsanguinarine

| Product | |
|---|---|
| | Carboxylic acid |
| | reagent |
| 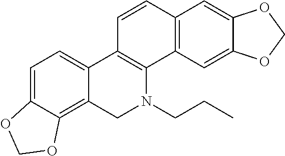 [SMILES: CCCN1Cc2c(ccc3c2OCO3)-c2ccc3cc4c(cc3c21)OCO4] | Propanoic acid |
| 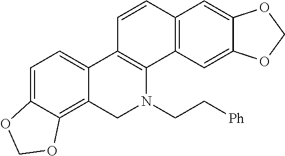 [SMILES: c1ccc(CCN2Cc3c(ccc4c3OCO4)-c3ccc4cc5c(cc4c32)OCO5)cc1] | Phenylacetic acid |
| | Carboxylic acid |
| 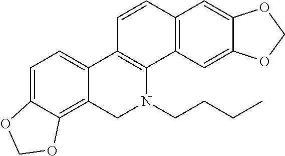 [SMILES: CCCCN1Cc2c(ccc3c2OCO3)-c2ccc3cc4c(cc3c21)OCO4] | Butanoic acid |
| 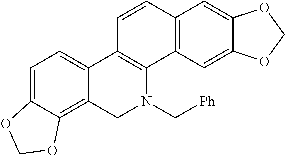 [SMILES: c1ccc(CN2Cc3c(ccc4c3OCO4)-c3ccc4cc5c(cc4c32)OCO5)cc1] | Benzoic acid |

Treatment of compound 15 with sodium borohydride in the presence of carboxylic acid results in reductive alkylation and formation of n-alkylated derivatives of dihydrosanguinarine.

### Example 4

### N-Alkyl Derivatives of Sanguinarine

N-alkyl derivatives of sanguinarine are prepared are prepared by treating a n-alkyl dihydrosanguinarine starting material using Jones oxidation conditions, chromium trioxide in dilute sulfuric acid and acetone.

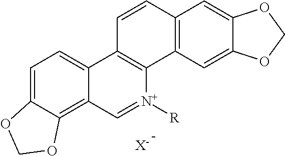

TABLE 2
Products resulting from oxiditation of n-alkyl dihydrosanguinarine starting material

| Product | Starting Material |
|---|---|
| 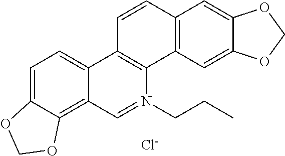 | 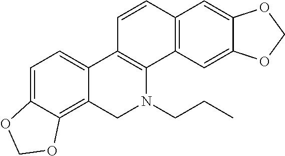 [SMILES: CCCN1Cc2c(ccc3c2OCO3)-c2ccc3cc4c(cc3c21)OCO4] |
| 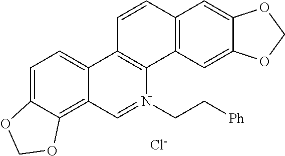 | 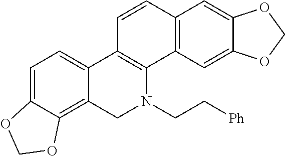 [SMILES: c1ccc(CCN2Cc3c(ccc4c3OCO4)-c3ccc4cc5c(cc4c32)OCO5)cc1] |
| 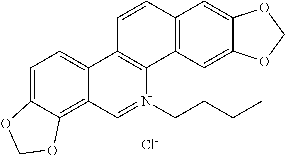 | 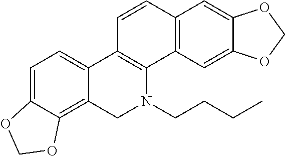 [SMILES: CCCCN1Cc2c(ccc3c2OCO3)-c2ccc3cc4c(cc3c21)OCO4] |
| 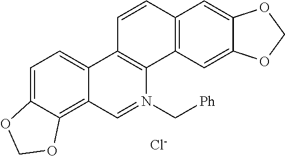 | 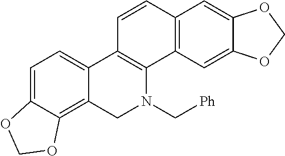 [SMILES: c1ccc(CN2Cc3c(ccc4c3OCO4)-c3ccc4cc5c(cc4c32)OCO5)cc1] |

Products from reductive alkylation of compound 15 are further reacted using chromium trioxide in dilute sulfuric acid and acetone to produce N-alkylated sanguinarine derivatives (Table 2).

### Example 5

### Alternative Synthesis of N-Alkyl Derivatives of Sanguinarine

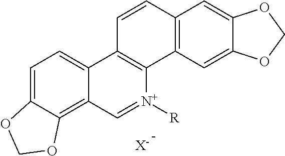

N-Alkyl derivatives of sanguinarine are prepared by treating norsanguinarine with alkyl halides. Table 1 shows N-alkyl derivatives of sanguinarine that are prepared from reacting norsanguinarine with alkyl halides.

TABLE 3
Products resulting from reacting various alkyl halides with norsanguinarine

| Product | |
|---|---|
| | Alkyl |
| | halide |
| 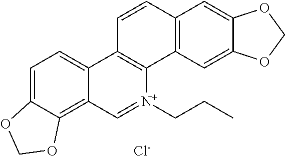 [SMILES: CCC[n+]1cc2c3c(ccc2c2ccc4cc5c(cc4c21)OCO5)OCO3 ; [Cl-]] | Chloro- propane |
| 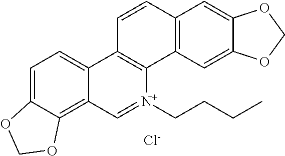 [SMILES: CCCC[n+]1cc2c3c(ccc2c2ccc4cc5c(cc4c21)OCO5)OCO3 ; [Cl-]] | Chloro- butane |
| 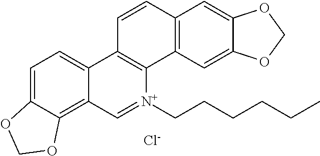 [SMILES: CCCCCC[n+]1cc2c3c(ccc2c2ccc4cc5c(cc4c21)OCO5)OCO3 ; [Cl-]] | Chloro- hexane |
| 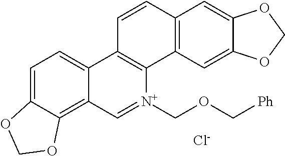 [SMILES: c1ccc(COC[n+]2cc3c4c(ccc3c3ccc5cc6c(cc5c32)OCO6)OCO4)cc1 ; [Cl-]] | Benzyl chloro- methyl ether |
| | Alkyl |
| | halide |
| | reactant |
| 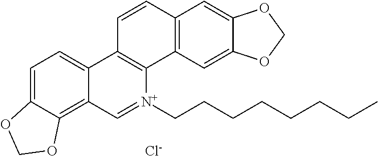 [SMILES: CCCCCCCC[n+]1cc2c3c(ccc2c2ccc4cc5c(cc4c21)OCO5)OCO3 ; [Cl-]] | Chloro- octane |
| 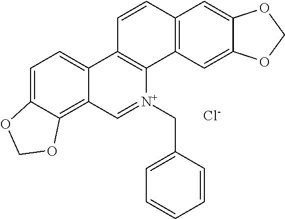 [SMILES: c1ccc(C[n+]2cc3c4c(ccc3c3ccc5cc6c(cc5c32)OCO6)OCO4)cc1 ; [Cl-]] | Benzyl- chloride |
| 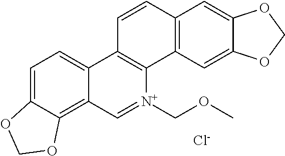 [SMILES: COC[n+]1cc2c3c(ccc2c2ccc4cc5c(cc4c21)OCO5)OCO3 ; [Cl-]] | Methoxy- methyl chloride |
| 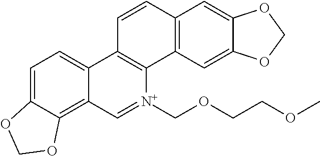 [SMILES: COCCOC[n+]1cc2c3c(ccc2c2ccc4cc5c(cc4c21)OCO5)OCO3] | 2- methoxy- ethoxy- methyl chloride |

### Example 6

### Alternative Synthesis of N-Alkyl Derivatives of Dihydronorsanguinarine

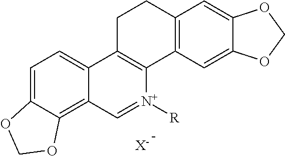

N-Alkyl derivatives of dihydronorsanguinarine are prepared by treating dihydronorsanguinarine with alkyl halides. Table 2 shows N-alkyl derivatives of dihydronorsanguinarine that are prepared from reacting dihydronorsanguinarine with alkyl halides.

TABLE 4
Products resulting from reacting various
alkyl halides with dihydronorsanguinarine

| Product | |
|---|---|
| | Alkyl |
| | halide |
| 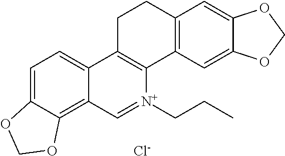 [SMILES: CCC[n+]1cc2c3c(ccc2c2c1-c1cc4c(cc1CC2)OCO4)OCO3 ; [Cl-]] | Chloro- propane |
| 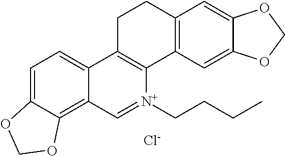 [SMILES: CCCC[n+]1cc2c3c(ccc2c2c1-c1cc4c(cc1CC2)OCO4)OCO3 ; [Cl-]] | Chloro- butane |
| 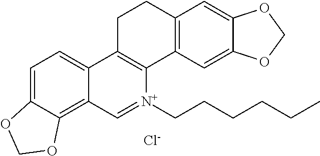 [SMILES: CCCCCC[n+]1cc2c3c(ccc2c2c1-c1cc4c(cc1CC2)OCO4)OCO3 ; [Cl-]] | Chloro- hexane |
| 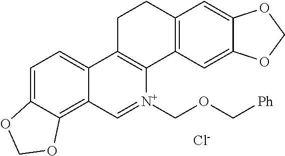 [SMILES: c1ccc(COC[n+]2cc3c4c(ccc3c3c2-c2cc5c(cc2CC3)OCO5)OCO4)cc1 ; [Cl-]] | Benzyl chloro- methyl ether |
| | Alkyl |
| | halide |
| | reactant |
| 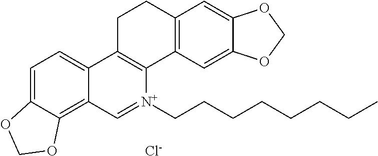 [SMILES: CCCCCCCC[n+]1cc2c3c(ccc2c2c1-c1cc4c(cc1CC2)OCO4)OCO3 ; [Cl-]] | Chloro- octane |
| 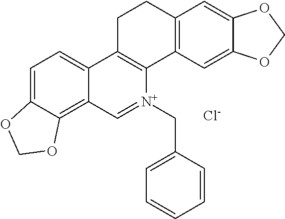 [SMILES: c1ccc(C[n+]2cc3c4c(ccc3c3c2-c2cc5c(cc2CC3)OCO5)OCO4)cc1 ; [Cl-]] | Benzyl- chloride |
| 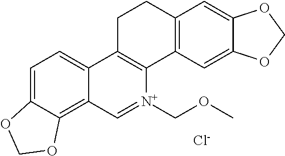 [SMILES: COC[n+]1cc2c3c(ccc2c2c1-c1cc4c(cc1CC2)OCO4)OCO3 ; [Cl-]] | Methoxy- methyl chloride |
| 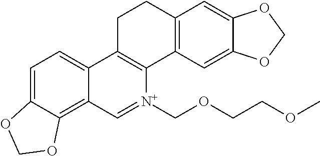 [SMILES: COCCOC[n+]1cc2c3c(ccc2c2c1-c1cc4c(cc1CC2)OCO4)OCO3] | 2- methoxy- ethoxy- methyl chloride |

### Example 7

### N-Ethyl Derivative of Sanguinarine as an Inhibitor of Cell Growth of Human Cancer Cell Line A549 (Lung Carcinoma)

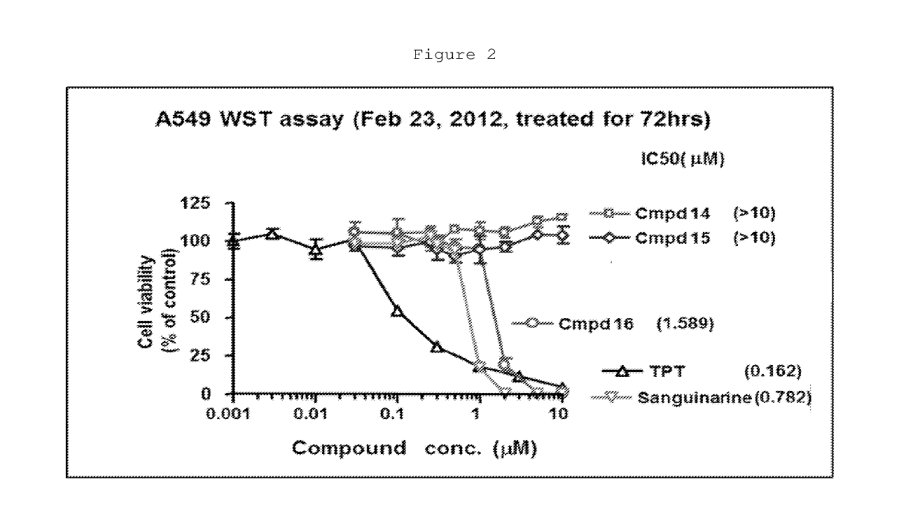

The N-ethyl derivative of sanguinarine inhibited cell growth of the human cancer cell line A549 (lung carcinoma) in vitro. A549 cells treated with the N-Ethyl derivative of sanguinarine showed decreased cell viability upon increased concentration of the N-ethyl derivative of sanguinarine. FIG. 2 shows the effect of the N-ethyl derivative of sanguinarine on human cancer cell line A549 (lung carcinoma).

### Example 8

### N-Alkyl Derivatives of Sanguinarine as an Inhibitor of Cell Growth of Human Cancer Cell Line A549 (Lung Carcinoma)

N-Alkyl derivatives of sanguinarine are inhibitors of cell growth in the human cancer cell line A549 (lung carcinoma) in vitro. A549 cells are treated with an N-alkyl derivative of sanguinarine and show decreased cell viability upon increased concentration of the N-alkyl derivative of sanguinarine.

### Example 9

### N-Ethyl Derivative of Sanguinarine as an Inhibitor of Cell Growth of Human Cancer Cell Line U87MG (Glioblastoma)

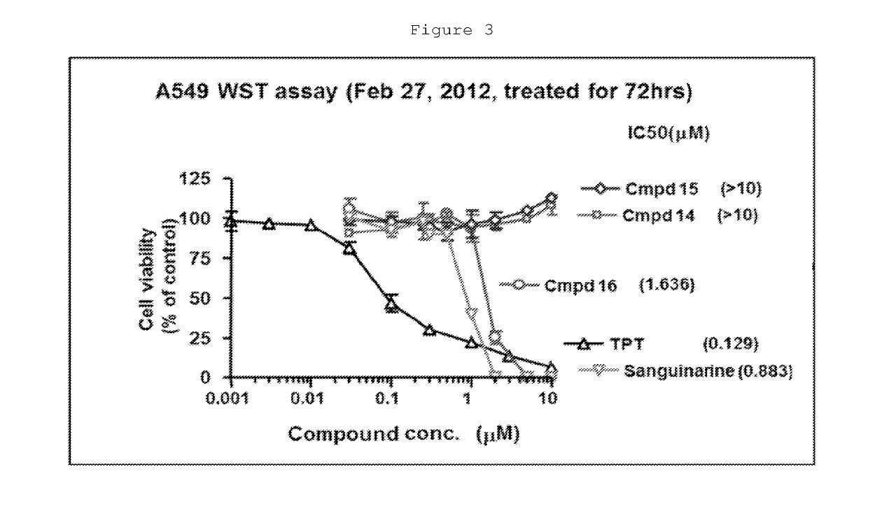

The N-ethyl derivative of sanguinarine inhibited cell growth of the human cancer cell line U87MG (glioblastoma) in vitro. U87MG cells treated with N-Ethyl derivative of sanguinarine showed decreased cell viability upon increased concentration of the N-ethyl derivative of sanguinarine. FIG. 3 shows the effect of the N-ethyl derivative of sanguinarine on human cancer cell line U87MG (glioblastoma).

### Example 10

### N-Alkyl Derivatives of Sanguinarine as an Inhibitor of Cell Growth of Human Cancer Cell Line U87MG (Glioblastoma)

N-Alkyl derivatives of sanguinarine are inhibitors of cell growth in the human cancer cell line U87MG (glioblastoma) in vitro. U87MG cells are treated with an N-alkyl derivative of sanguinarine and show decreased cell viability upon increased concentration of the N-alkyl derivative of sanguinarine.

### Discussion

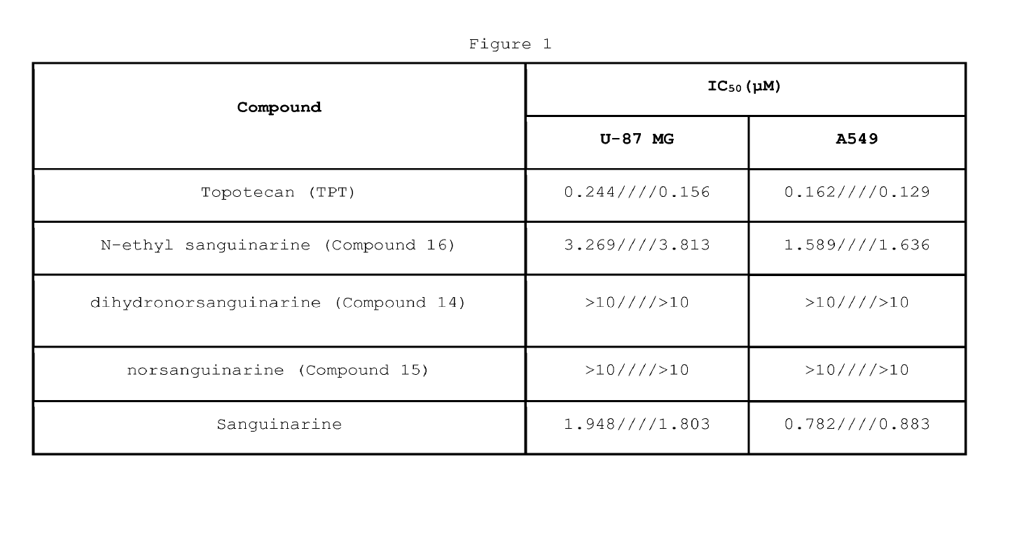

The ability of sanguinarine, norsanguinarine (Compound 15), dihydronorsanguinarine (Compound 14), and the ethyl-substituted derivative of sanguinarine (Compound 17) to inhibit growth in vitro of two human cancer cell lines, U87MG (glioblastoma) and A549 (lung carcinoma), was analyzed. A table summarizing the results is shown in FIG. 1. The ethyl-substituted derivative of sanguinarine (Compound 17) inhibited both cell lines with an ICH of about 3.3 and 1.6 uM respectively, whereas sanguinarine itself had ICH values of 2.0 and 0.8 uM, respectively. Sanguinarine, norsanguinarine (Compound 15), dihydronorsanguinarine (Compound 14), and the ethyl-substituted derivative of sanguinarine (Compound 17) were also compared to the known, highly active, anti-cancer agent Topotecan which showed ICH values of 0.3 and 0.2 uM against the same cell lines. Dihydrosanguinarine and norsanguinarine had no activity up to 10 uM. The results show that N-alkyl derivatives of sanguinarine are inhibitors of cell growth of human cancer cells.

The results show that N-substituted derivatives of sanguinarine inhibit cell growth and reduce cell viability of cancer cells, including lung cancer cells and glioblastoma cells.

N-substituted derivatives of sanguinarine may be synthesized by a general method of which one embodiment is exemplified in Example 1. Those of skill in the art will readily understand that the method of Example 1 may be generalized to permit the synthesis of analogs and derivatives of sanguinarine. Said analogues and derivatives may be substituted at other positions of the aromatic rings. One skilled in the art would additionally understand that the process described exemplified by the synthetic route in Scheme 1 may be modified by using derivatives of dicyano compound 6 and iodo compound 10. Modified versions of compound 6 and compound 10 may be achieved by functionalizing the aromatic ring of any of compounds 1-5 and 7-9 or purchasing commercially available reagents which may be used in place of any of compounds 1-10. Techniques for functionalizing an aromatic ring are readily known to those having ordinary skill in the art as described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references herein, which are incorporated by reference herein.

### REFERENCES

Aburai N, Yoshida M, Ohnishi M, Kimura, K-I (2010). Sanguinarine as a potent and specific inhibitor of protein phosphatase 2C in vitro and induces apotosis via phosphorylation of p38 in HL60 cells. Biosci. Biotechnol. Biochem. 74 (3):548-552.
Ahmad N, Gupta S, Husain M M, Heiskanen K M, Mukhtar H. (2000). Differential antiproliferative and apoptotic response of sanguinarine for cancer cells versus normal cells. Clinical Cancer Research 6:1524-1258.
Lammers T and Lavi S. (2007). Role of type 2C protein phosphatases in growth regulation and in cellular stress signaling. Critical Reviews in Biochemistry and Molecular Biology 42:437-461.
Samudrala R, Gupta R C, Johnson F. (2011) Total synthesis of norsanguinarine and various sanguinarine analogues. Confidential report to Lixte Biotechnology, Inc.
Shreeram S and Bulavin D W. (2008). PPM1H. Cancer Biology & Therapy 7(2):293-294.
Sugiura T, Noguchi W, Sakurai K, Hattori C. (2008). Cancer Biology & Therapy 7(2):285-292.
Sun M, Liu C, Nadiminty N, Lou W, Zhu Y, Yang J, Evans C P, Zhou Q, Gao A C. (2012). Inhibition of Stat3 activation by sanguinarine suppresses prostate cancer cell growth and invasion. The Prostate 72:82-89.
Chuman Y, Yagi H, Fukuda T, Nomura T, Matsukizono M, Shimohigashi Y, Sakaguchi K. (2008). Characterization of the active site and a unique uncompetitive inhibitor of the PPM1-type protein phosphatase PPM1D. Protein & Peptide Letters 15:938-948.
Iyer S, Younker J M, Czyrych P J, Hengge A C. (2004). Bioorganic & Medicinal Chemistry Letters 14:5931-5935.
Rayter S, Elliott R, Travers J, Rowlands M G, Richardson T B, Boxall K, Jones K, Linardopoulos S, Workman P, Aherne W, Lord C J, Ashworth A. (2008). Oncogene 27:1036-1044.
Swierczek K, Pandey A S, Peters J W, Hengge A C. (2003). A comparison of phosphonothioic acids with phosphonic acids as phosphatase inhibitors. J. Med. Chem. 46:3703-3708.
Rogers J P, Beuscher I V, A E, Flajolet M, McAvoy T, Nairn A C, Olson A J, Greengard P (2006). Discovery of protein phosphatase 2C inhibitors by virtual screening. J Med Chem 49:1658-1667.
Saurabh Shahane, Fadila Louafi, Julie Moreau, Jean-Pierre Hurvois, Jean-Luc Renaud, Pierre van de Weghe, and Thierry Roisnel Eur. J. Org. Chem. 2008, 4622-4631
Johnson, Francis and Nasutavicus, W. J. Org. Chem., 1962, 27, 3953-3958 Yuhsuke Ishihara, Shuhei Azuma, Tominari Choshi, Kakujirou Kohno, Kanako Ono, Hiroyuki Tsutsumi, Takashi Ishizu, Satoshi Hibino Tetrahedron 2011, 67, 1320-1333
Ishii H, Ishikawa T, Ichikawa Y-I, Sakamoto M, Ishikawa M, Takahashi T. Chem Pharm Bull 1984, 32, 2984-2994

1. A compound having the structure: 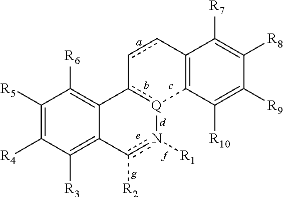
wherein:
bond “a” is a single bond or a double bond;
bond “b” is a single bond or a double bond;
bond “c” is a single bond which is present or absent;
bond “d” is a single bond which is present or absent;
bond “e” is a single bond, a double bond or a triple bond;
bond “f” is a single bond which is present or absent;
bond “g” is a single bond which is present or absent;
Q is 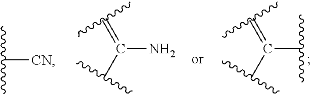
R1 is present or absent, wherein when R1 is present, then bond “f” is present, and when R1 is absent, then bond “f” is absent;
R2 is present or absent, wherein when R2 is present, then bond “g” is present, and when R2 is absent, then bond “g” is absent;
wherein when bond “d” is absent, then bond “a” is a single bond, bond “b” is a single bond, bond “c” is absent, bond “e” is a triple bond, R1 is absent, R2 is absent and Q is 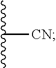 [SMILES: *C#N]
wherein when bond “d” is present, then bond “b” is a double bond, bond “e” is a single bond or a double bond and R2 is present;
wherein when bond “c” is absent and bond “d” is present, then bond “a” is a single bond, “e” is a double bond and Q is 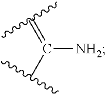 [SMILES: *C(=*)N]
wherein when bond “c” is present, than bond “e” is a single bond or a double bond and Q is 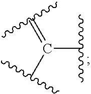 [SMILES: *C(*)=* ; ";"]
wherein when bond “e” is a single bond, then bond “a” is a double bond and R1 is present;
wherein when R1 is present, then N is N⁺ and R1 is C1-C10 alkyl other than methyl, C2-C10 alkenyl, C3-C10 alkynyl, C3-C7 cycloalkyl, C2-C10 acyl, C2-C10 heteroalkyl, aryl or arylmethyl, any one of which is unsubstituted or substituted at one or more positions with halogen, C1-C5 alkyl, C1-C5 heteroalkyl, C2-C7 acyl, C3-C7 cycloalkyl or aryl;
wherein when R2 is present, then R2 is hydrogen, halogen or amino, wherein the amino is unsubstituted or substituted with one or more C1-C5 alkyl, C2-C7 acyl, aryl or arylmethyl;
wherein R3, R4, R5 and R6 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R3, R4, R5 and R6 are other than hydrogen, or R3 and R4 are linked so as to form —O—(CX₂)ₙ—O—, or R4 and R5 are linked so as to form —O—(CX₂)ₙ—O—, or R5 and R6 are linked so as to form —O—(CX₂)ₙ—O—, wherein X is hydrogen, methyl or fluorine and n is 1-2;
wherein R7, RB, R9 and R10 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R7, R8, R9 and R10 are other than hydrogen, or R7 and R8 are linked so as to form —O—(CX₂)ₙ—O—, R8 and R9 are linked so as to form —O—(CX₂)ₙ—O—, or R9 and R10 are linked so as to form —O—(CX₂)ₙ—O—, wherein X is hydrogen, methyl or fluorine and n is 1-2; and
wherein when bond “c” is present and R2 is hydrogen, then R1 is present, or
a pharmaceutically acceptable salt thereof.

2. (canceled)

3. The compound according to claim 1, having the structure:

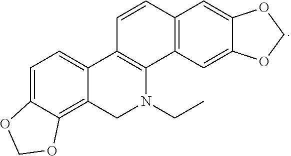 [SMILES: CCN1Cc2c(ccc3c2OCO3)-c2ccc3cc4c(cc3c21)OCO4]

4. The compound according to claim 1, having the structure: 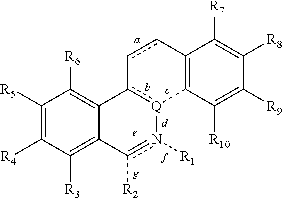
wherein:
bond “a” is a single bond or a double bond;
bond “b” is a single bond or a double bond;
bond “c” is a single bond which is present or absent;
bond “d” is a single bond which is present or absent;
bond “e” is a double bond or a triple bond;
bond “f” is a single bond which is present or absent;
bond “g” is a single bond which is present or absent;
Q is 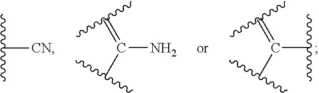
R1 is present or absent, wherein when R1 is present, then bond “f” is present, and when R1 is absent, then bond “f” is absent;
R2 is present or absent, wherein when R2 is present, then bond “g” is present, and when R2 is absent, then bond “g” is absent;
wherein when bond “d” is absent, then bond “a” is a single bond, bond “b” is a single bond, bond “c” is absent, bond “e” is a triple bond, R1 is absent, R2 is absent and Q is 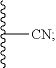 [SMILES: *C#N]
wherein when bond “d” is present, then bond “b” is a double bond, bond “e” is a double bond and R2 is present;
wherein when bond “c” is absent and bond “d” is present, then bond “a” is a single bond and Q is 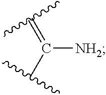 [SMILES: *C(=*)N]
wherein when bond “c” is present, then Q is 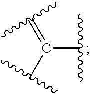 [SMILES: *C(*)=*]
wherein when R1 is present, then N is N⁺ and R1 is C1-C10 alkyl other than methyl, C2-C10 alkenyl, C3-C10 alkynyl, C3-C7 cycloalkyl, C2-C10 acyl, C2-C10 heteroalkyl, aryl or arylmethyl, any one of which is unsubstituted or substituted at one or more positions with halogen, C1-C5 alkyl, C1-C5 heteroalkyl, C2-C7 acyl, C3-C7 cycloalkyl or aryl;
wherein when R2 is present, then R2 is hydrogen, halogen or amino, wherein the amino is unsubstituted or substituted with one or more C1-C5 alkyl, C2-C7 acyl, aryl or arylmethyl;
wherein R3, R4, R5 and R6 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R3, R4, R5 and R6 are other than hydrogen, or R3 and R4 are linked so as to form —O—(CX₂)ₙ—O—, or R4 and R5 are linked so as to form —O—(CX₂)ₙ—O—, or R5 and R6 are linked so as to form —O—(CX₂)ₙ—O—, wherein X is hydrogen, methyl or fluorine and n is 1-2;
wherein R7, R8, R9 and R10 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R7, RB, R9 and R10 are other than hydrogen, or R7 and RB are linked so as to form —O—(CX₂)ₙ—O—, R8 and R9 are linked so as to form —O—(CX₂)ₙ—O—, or R9 and R10 are linked so as to form —O—(CX₂)ₙ—O—, wherein X is hydrogen, methyl or fluorine and n is 1-2; and
wherein when bond “c” is present and R2 is hydrogen, then R1 is present, or
a pharmaceutically acceptable salt thereof.

5. The compound according to claim 4, having the structure: 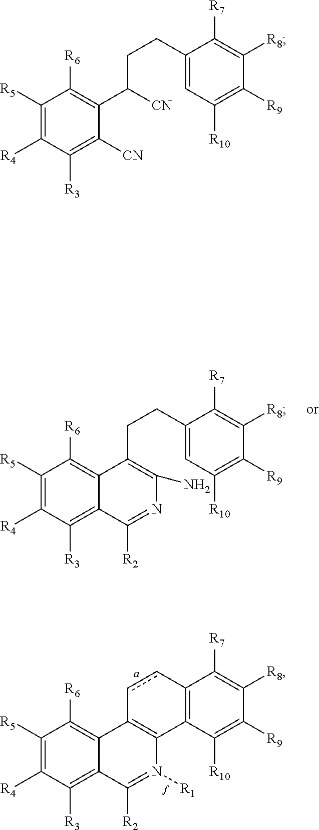
or
a pharmaceutically acceptable salt thereof.

6. (canceled)

7. The compound according to claim 1 having the structure: 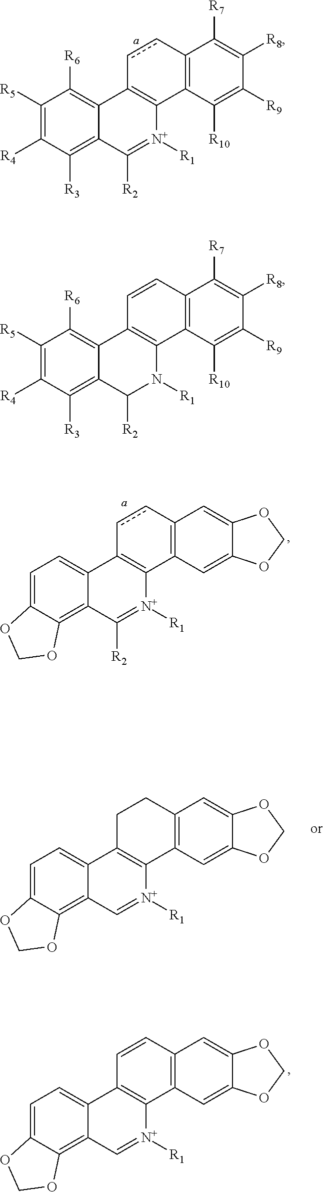
wherein R1 is present, or
a pharmaceutically acceptable salt thereof.

8. The compound according to claim 2, wherein R8 and R9 are linked so as to form —O—CH₂—O— and/or wherein R3 and R4 are linked so as to form —O—CH₂—O—.

9. (canceled)

10. The compound according to claim 2, wherein at least two of R3, R4, R5 and R6 are other than hydrogen; and at least two of R7, R8, R9 and R10 are other than hydrogen; or R5, R6, R7 and R10 are hydrogen.

11.-16. (canceled)

17. The compound according to claim 7, wherein R1 is C1-C10 alkyl other than methyl, which is unsubstituted or substituted at one or more positions with halogen, C1-C5 alkyl, C1-C5 heteroalkyl, C2-C7 acyl, C3-C7 cycloalkyl or aryl.

18. (canceled)

19. (canceled)

20. The compound according to claim 17, wherein R1 is ethyl, propyl, butyl, pentyl, hexyl, heptyl, octyl, nonyl or decyl.

21. (canceled)

22. The compound according to claim 17, wherein the compound is a pharmaceutically acceptable salt.

23. The compound according to claim 22, wherein the pharmaceutically acceptable salt is a chloride, iodide, bromide, sulfate, bisulfate, nitrate, phosphate, sulfonate, formate, tartrate, maleate, malate, citrate, benzoate, acetate, valerate, oleate, palmitate, stearate, laurate, salicylate, ascorbate, tosylate, fumarate, succinate, napthylate, mesylate, glucoheptonate, lactobionate, laurylsulphonate or phenoate salt.

24. (canceled)

25. The compound according to claim 1, having the structure:

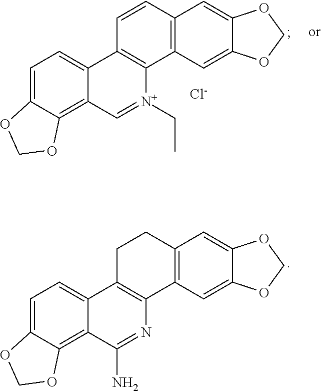 [SMILES: CC[n+]1cc2c3c(ccc2c2ccc4cc5c(cc4c21)OCO5)OCO3 ; [Cl-] ; Nc1nc2c(c3ccc4c(c13)OCO4)CCc1cc3c(cc1-2)OCO3]

26. (canceled)

27. (canceled)

28. The compound according to claim 1, having the structure:

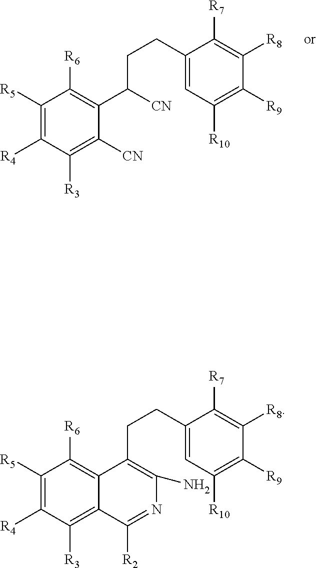

29. (canceled)

30. (canceled)

31. The compound according to claim 1, having the structure:

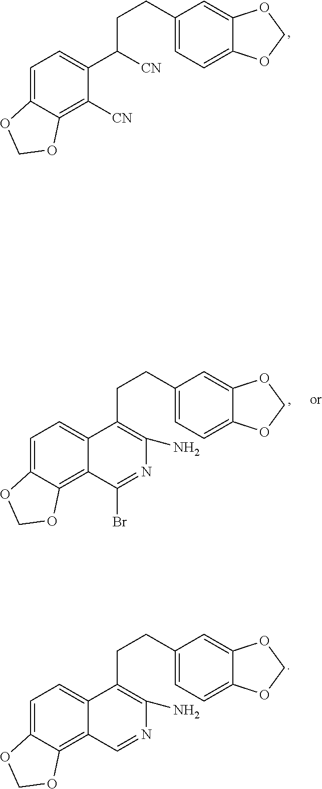 [SMILES: N#Cc1c(C(C#N)CCc2ccc3c(c2)OCO3)ccc2c1OCO2 ; Nc1nc(Br)c2c3c(ccc2c1CCc1ccc2c(c1)OCO2)OCO3 ; Nc1ncc2c3c(ccc2c1CCc1ccc2c(c1)OCO2)OCO3]

32. A pharmaceutical composition comprising the compound of claim 1 and a pharmaceutically acceptable carrier.

33. A method of inhibiting protein phosphatase 2C (PP2C) or inhibiting growth of cancer cells comprising contacting the PP2C or cancer cells with a compound having the structure: 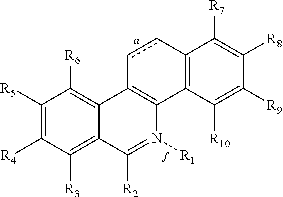
wherein bond “a” is a single bond or a double bond;
bond “f” is a single bond which is present or absent;
R1 is present or absent, wherein when R1 is present, then bond “f” is present, and when R1 is absent, then bond “f” is absent;
wherein when R1 is present, then N is N⁺ and R1 is C1-C10 alkyl other than methyl, C2-C10 alkenyl, C3-C10 alkynyl, C3-C7 cycloalkyl, C2-C10 acyl, C2-C10 heteroalkyl, aryl or arylmethyl, any one of which is unsubstituted or substituted at one or more positions with halogen, C1-C5 alkyl, C1-C5 heteroalkyl, C2-C7 acyl, C3-C7 cycloalkyl or aryl;
wherein when R2 is present, then R2 is hydrogen, halogen or amino,
wherein the amino is unsubstituted or substituted with one or more C1-C5 alkyl, C2-C7 acyl, aryl or arylmethyl;
wherein R3, R4, R5 and R6 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R3, R4, R5 and R6 are other than hydrogen, or R3 and R4 are linked so as to form —O—(CX₂)ₙ—O—, or R4 and R5 are linked so as to form —O—(CX₂)ₙ—O—, or R5 and R6 are linked so as to form —O—(CX₂)ₙ—O—, wherein X is hydrogen, methyl or fluorine and n is 1-2;
wherein R7, R8, R9 and R10 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R7, R8, R9 and R10 are other than hydrogen, or R7 and R8 are linked so as to form —O—(CX₂)ₙ—O—, R8 and R9 are linked so as to form —O—(CX₂)ₙ—O—, or R9 and R10 are linked so as to form —O—(CX₂)ₙ—O—, wherein X is hydrogen, methyl or fluorine and n is 1-2; and
wherein when bond “c” is present and R2 is hydrogen, then R1 is present, or
a pharmaceutically acceptable salt thereof, so as to thereby inhibit the PP2C or the growth of the cancer cells.

34.-36. (canceled)

37. The method of claim 33, wherein the cancer is lung cancer, breast cancer, prostate cancer, cervical cancer, pancreatic cancer, colon cancer, ovarian cancer; stomach cancer, esophagus cancer, mouth cancer, tongue cancer, gum cancer, skin cancer, muscle cancer, heart cancer, liver cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, gall bladder cancer, ocular cancer, joint cancer, glioblastoma, lymphoma, or leukemia.

38. The method of claim 33, wherein the compound inhibits the growth of cancer cells to a greater extent than normal cells.

39. A method of treating cancer in a patient afflicted by the cancer, comprising administering to the patient a compound having the structure: 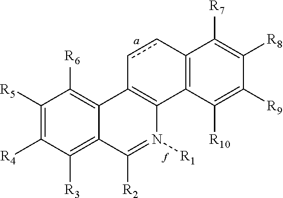
wherein bond “a” is a single bond or a double bond;
bond “f” is a single bond which is present or absent;
R1 is present or absent, wherein when R1 is present, then bond “f” is present, and when R1 is absent, then bond “f” is absent;
wherein when R1 is present, then N is N⁺ and R1 is C1-C10 alkyl other than methyl, C2-C10 alkenyl, C3-C10 alkynyl, C3-C7 cycloalkyl, C2-C10 acyl, C2-C10 heteroalkyl, aryl or arylmethyl, any one of which is unsubstituted or substituted at one or more positions with halogen, C1-C5 alkyl, C1-C5 heteroalkyl, C2-C7 acyl, C3-C7 cycloalkyl or aryl;
wherein when R2 is present, then R2 is hydrogen, halogen or amino, wherein the amino is unsubstituted or substituted with one or more C1-C5 alkyl, C2-C7 acyl, aryl or arylmethyl;
wherein R3, R4, R5 and R6 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R3, R4, R5 and R6 are other than hydrogen, or R3 and R4 are linked so as to form —O—(CX₂)ₙ—O—, or R4 and R5 are linked so as to form —O—(CX₂)ₙ—O—, or R5 and R6 are linked so as to form —O—(CX₂)ₙ—O—, wherein X is hydrogen, methyl or fluorine and n is 1-2;
wherein R7, R8, R9 and R10 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R7, R8, R9 and R10 are other than hydrogen, or R7 and R8 are linked so as to form —O—(CX₂)ₙ—O—, R8 and R9 are linked so as to form —O—(CX₂)ₙ—O—, or R9 and R10 are linked so as to form —O—(CX₂)ₙ—O—, wherein X is hydrogen, methyl or fluorine and n is 1-2; and
wherein when bond “c” is present and R2 is hydrogen, then R1 is present, or
a pharmaceutically acceptable salt thereof, so as to thereby treat the cancer.

40. (canceled)

41. (canceled)

42. (canceled)

43. A process of preparing the compound of claim 1 comprising:
(a) reacting a first compound with a strong base and a second compound;
wherein the first compound has the structure: 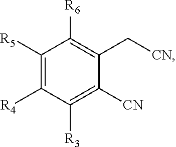
wherein R3, R4, R5 and R6 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R3, R4, R5 and R6 are other than hydrogen, or R3 and R4 are linked so as to form —O—(CX₂)ₙ—O—, or R4 and R5 are linked so as to form —O—(CX₂)ₙ—O—, or R5 and R6 are linked so as to form —O—(CX₂)ₙ—O—, wherein X is hydrogen, methyl or fluorine and n is 1-2;
wherein when R3, R4, R5 or R6 are hydroxyl, thiol or amino, R3, R4, R5 or R6 are optionally substituted with a suitable protecting group; and
wherein the second compound has the structure: 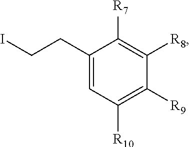
wherein R7, R8, R9 and R10 are each independently hydrogen, hydroxyl, C1-C5 alkoxy, C2-C7 acyloxy, aryloxy, arylmethyloxy, thiol, C1-C5 alkylthiol, C2-C7 acylthiol, arylthiol, arylmethylthiol, amino, C1-C5 monoalkylamino, C1-C5 dialkylamino, C2-C7 acylamino or arylmethylamino, wherein at least one of R7, R8, R9 and R10 are other than hydrogen, or R7 and R8 are linked so as to form —O—(CX₂)ₙ—O—, R8 and R9 are linked so as to form —O—(CX₂)ₙ—O—, or R9 and R10 are linked so as to form —O—(CX₂)ₙ—O—, wherein X is hydrogen, methyl or fluorine and n is 1-2;
wherein when R7, R8, R9 or R10 are hydroxyl, thiol or amino, R7, R8, R9 or R10 are optionally substituted with a suitable protecting group;
to form a product having the structure: 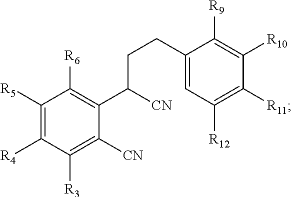
(b) reacting the product of step (a) with an acid to form a product having the structure: 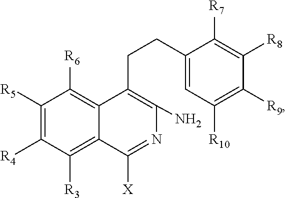
wherein X is Cl, Br, I or trifluoromethanesulfonate;
(c) reacting the product of step (b) under hydrogenation conditions to form a product having the structure: 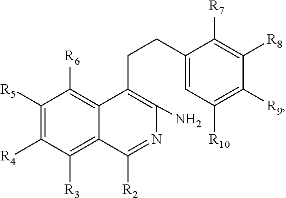
wherein R2 is hydrogen;
(d) reacting the product of step (c) with sodium nitrite and acid, then heating to form a product having the structure: 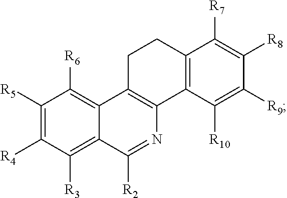
(e) reacting the product of step (d) with platinum or palladium, and heating to form a product having the structure: 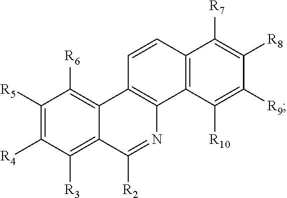
and
(ee) reacting the product of step (e) with a compound having the structure:
LG-R₁,
wherein LG is a leaving group; and
wherein R1 is C1-C10 alkyl other than methyl, C2-C10 alkenyl, C3-C10 alkynyl, C3-C7 cycloalkyl, C2-C10 acyl, C2-C10 heteroalkyl, aryl or arylmethyl, any one of which is unsubstituted or substituted at one or more positions with halogen, C1-C5 alkyl, C1-C5 heteroalkyl, C2-C7 acyl, C3-C7 cycloalkyl or aryl;
to form a product having the structure: 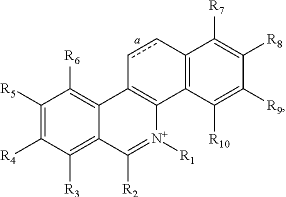
wherein bond “a” is a double bond.

44.-58. (canceled)
